(19) United States
(12) Patent Application Publication
(10) Pub. No.: US 2024/0215613 A1
(43) Pub. Date: Jul. 4, 2024

(54) SINGLE CELL PROTEIN PRODUCTS CONTAINING METHANOL-FED METHYLOTROPHS AND USES THEREOF

(71) Applicants: DeNova, Inc., Halifax, Nova Scotia (CA); NATIONAL RESEARCH COUNCIL OF CANADA, Ottawa (CA)

(72) Inventors: Brianna STRATTON, Halifax, Nova Scotia (CA); Sean TIBBETTS, Halifax, Nova Scotia (CA); Marta PIERCEY, Halifax, Nova Scotia (CA)

(21) Appl. No.: 18/560,057

(22) PCT Filed: May 14, 2022
(86) PCT No.: PCT/IB2022/054504

§ 371 Date: Nov. 9, 2023

Related U.S. Application Data
(60) Provisional application No. 63/188,687, filed on May 14, 2021.

(51) Int. Cl. A23K 10/16; A23K 10/22; A23K 10/24; A23K 10/26; A23K 10/30; A23K 20/142; A23K 20/174; A23K 20/22; A23K 50/80

(52) U.S. Cl. CPC A23K 10/16; A23K 10/22; A23K 10/24; A23K 10/26; A23K 10/30; A23K 20/142; A23K 20/174; A23K 20/22; A23K 50/80

(57) ABSTRACT

Provided herein are methylotrophic single cell protein products as a source of protein for use in a variety of methods. Also provided are animal feeds comprising the methylotrophic single cell protein products and methods of making and using the methylotrophic single cell protein products.

### BACKGROUND

Growth in human and animal populations has put a strain on global feed production systems. For example, there are insufficient resources to produce food required by the rapidly expanding aquaculture industry, which produces sea and freshwater organisms for consumption. These organisms include fish, a primary source of energy and nutrients for humans and animals. Global aquaculture production surpassed 114 million tonnes in 2018, 82 million tonnes of which came from aquatic animals (FAO, 2020). Aquatic animal aquaculture production is projected to reach 109 million tonnes in 2030, an increase of 32 percent (26 million tonnes) over 2018 (FAO, 2020).

Fish require a diet that is rich in high-quality protein. Obtaining or producing this protein from fish meal, fish oil, and other marine protein sources is often expensive, resource-intensive, and environmentally burdensome. Plant proteins are heavily used in modern aquafeeds, but are incomplete in their essential amino acid (EAA) profile; especially key EAAs like methionine and lysine. Single cell protein (SCP) products derived from yeasts, algae and bacteria offer an alternative to animal and plant proteins but large-scale development and adoption of SCP meals requires substantial evaluation to ensure the SCP products are safe, nutritious, and economically feasible. Also of critical importance is whether fish will consume SCP containing feed, what proportions of feed can constitute SCP, and how digestible the amino acids from the SCP prove to be. Nonetheless, novel affordable and sustainable protein sources are needed to address challenges presented by protein scarcity.

### BRIEF SUMMARY

Inexpensive sources of protein are necessary to meet the needs of growing human and animal populations. SCP products offer such an alternative but require a carbon source to divide and create a biomass until proper culture conditions. Methanol can be a low cost carbon source for single cell organisms that is available in large quantities but is known to be toxic, even to organisms that utilize methanol for growth. Methylotrophs are a diverse group of microorganisms that can use reduced one-carbon compounds, such as methanol or methane, as the carbon source for their growth. Herein are methylotrophic single cell protein products and methods of making them and methods of using them in animal feed. Also provided are animal feeds comprising the methylotrophic single cell protein products.

### BRIEF DESCRIPTION OF THE DRAWINGS

FIG. 1 is a graph showing growth of juvenile Atlantic salmon (Salmo salar) fed five nutritionally-balanced experimental test diets containing varying dietary inclusion of spray-dried (SD) and freeze-dried (FD) J25 SCP meals.

FIG. 2 is a graph showing growth of Atlantic salmon (Salmo salar L.) fed four nutritionally-balanced experimental test diets containing varying dietary inclusion of spray-dried J25 SCP meal during the seawater grower phase.

FIG. 3 is a bar graph showing the essential amino acid, essential n-3 LC-PUFA (EPA+DHA) and phosphorous content of the four nutritionally-balanced experimental test diets fed to Atlantic salmon (Salmo salar L.) during the seawater grower phase in relation to their published dietary requirements (NRC 2011). The test diets contain more essential amino acid, essential n-3 LC-PUFA (EPA+DHA) and phosphorous content than the published dietary requirements.

FIG. 4 is a graph showing the result from the fillet qualitative descriptive analysis (QDA) evaluation of Atlantic salmon (Salmo salar L.) fed four nutritionally-balanced experimental test diets containing varying dietary inclusion of spray-dried J25 SCP meal during the seawater grower phase.

### DETAILED DESCRIPTION

More than half of global seafood supplies are farmed, making aquaculture a significant source of low GHG-emitting aquatic animal protein for human consumption (Poore and Nemecek 2018; Edwards et al. 2019; FAO 2020, Tacon et al. 2020). As the most rapidly growing food production sector globally, the aquaculture industry has a market value of about $250 billion USD (FAO 2020), with farmed salmon accounting for $18 billion (FAO 2020). Salmon have high protein requirements, and their feeds account for about half of operating costs in salmon farming. Faced with tremendous demand, salmon aquaculture faces challenges associated with the ubiquitous need to source sufficient quantities of nutritionally-compatible, economical and sustainable protein feedstocks as supplements to more judicious use of finite marine resources (Gatlin et al. 2007; Naylor et al. 2009; Tacon and Metian 2015). This has led to remarkable downward trends in Fish In: Fish Out (FIFO) ratios (Tacon and Metian 2008; Kaushik and Troell 2010; Kok et al. 2020), due to expanding utilization of less-costly terrestrial feed inputs; predominantly soy and corn (Foroutani et al. 2018, 2020). However, broad utilization of plant-based ingredients at high levels is challenging due to poor palatability, indigestible fibers, intestinal health impacts, presence of anti-nutritional factors (ANFs), interferences with flesh pigmentation and questionable ecological-sustainability (Young and Pellett 1994; Oliva-Teles et al. 2015; Pahlow et al. 2015; Fry et al. 2016; Turchini et al. 2019; Tzachor 2019). Plant-proteins heavily used in modern aquafeeds are also incomplete in their essential amino acid (EAA) profile; especially key EAAs like methionine (soy) and lysine (corn).

SCP products show promise as sustainable sources of high-quality protein (Matassa et al. 2016; Ritala et al. 2017; Tibbetts 2018; Couture et al. 2019; Cottrell et al. 2020; Jones et al. 2020). SCP are derived from prokaryotic and/or eukaryotic microorganisms, i.e., microscopic single cell organisms such as bacteria, archaea and eubacteria of all species, as well as yeast and fungi. As used herein, the biomass of single-celled microorganisms, whether further processed or not, can be used as a protein-containing food source or food ingredient and is produced by cultivating microorganisms on substrates that may include hydrocarbons, alcohols, or waste products. The single cell protein produced can be a nutrient source for aquaculture, agriculture, animals or humans. Such products may be capable of replacing or supplementing conventional protein-rich ingredients commonly used in animal feeds, including farmed salmon feeds.

Feeds produced from SCP are attractive alternatives to conventional sources from a production, ecological and physical footprint standpoint, as they can be intensively produced under highly controlled conditions in enclosed bioreactors free from environmental stressors (e.g., temperature fluctuations, unpredictable climatic condition, droughts or floods, invasive contamination, etc.). For example, a 40.5 hectare SCP facility is capable of generating the same protein production as a 4,047 hectare soybean operation (Tlusty et al. 2017) and a ton of bacterial SCP meal can be produced with a 20 to 140 times lower freshwater footprint than a ton of fish meal or soy protein concentrate (Matassa et al. 2016). Although the mass industrial production and scale up of bacteria-derived SCP is still in its initial stages (Fasolin et al. 2019), sources of high-protein SCP meals used in animal and aquaculture feeds have shown encouraging production results and, as such, is not a new area of interest for salmonid nutrition (Beck et al. 1979; Bergstrom 1979). For large-scale development and adoption of SCP meals in aquaculture feeds, however, more sources must be established and evaluated for their safety, nutritional value and economic suitability in aquafeed production. Additionally, within each target species, the effects of the novel SCP ingredients must be evaluated for their acceptable inclusion rates and effects on feed consumption (e.g., organoleptic properties), digestibility, nutrient bioavailability, production performance, fish health, final product quality and production economics. Of particular importance, is the establishment of the species-specific apparent digestibility coefficients (ADCs) for their constituent nutrients when incorporated into species level aquafeeds; which are requisite for on-going formulation of research diets and commercial feeds.

SCP can be a useful and inexpensive food source. Production process include growth of a biomass, i.e., cellular material of the microorganisms, in a medium containing a carbon source. Many single cell protein production processes have included the use of methane as the sole source of carbon for microorganism growth because it can be available in large quantities and at low cost. Certain microorganisms are methylotrophic and can grow in and bioconvert (i.e., use as a carbon and energy source) methanol. For example, such a microorganism is one that can grow and reproduce in a medium containing methanol; for example, in a medium containing about 10% w/vol. Comparative methanol tolerance may be determined by growth rate, productivity, or cell concentration.

Although methanol has been used on occasion as the primary or sole carbon source, such processes to date often have low productivity (biomass (g)/media (L)/time (h)). A number of factors contribute to how much single cell protein a process can produce in a period of time when methanol is used as the carbon source. Increasing the biomass (g/L) in a methanol-fed SCP production system requires high methanol input, but methanol is known to be toxic to bacterial cells (Ebbinghaus et al., 1981 The Production of Single Cell Protein from Methanol by Bacteria. Moo-Young, M. (Ed). Advances in Biotechnology Volume II Fuels, Chemicals, Foods and Waste Treatment. Elsevier Science Publishers). Thus, yield (efficient conversion of methanol to biomass) may be reduced with higher methanol concentrations. Similarly, certain methylotrophic organisms can utilize methanol as their sole carbon source but often show lower productivity at high concentrations of methanol. Thus, it can be challenging to use methylotrophic organisms to achieve a high growth rate and a high protein content, with desirable essential amino acid profiles and ADCs.

Provided herein are animal feeds in which all or part of the animal-derived protein and/or plant-based protein products are replaced by SCP. As used herein, animal-derived protein product refers to products derived from an animal and having a crude protein content greater than 60%. Examples of animal-derived protein products include, but are not limited to, feather meal, blood meal, poultry by-products, and fish meal. As used herein, plant-based protein product refers to a plant based product, including isolates and concentrates with a crude protein content of 40% or greater. Examples of plant-based protein products include, but are not limited to, soy-based protein products, corn-based protein products, wheat-based protein products, guar proteins, legume-based protein products such as pea-based protein products, and barley-based protein products.

The SCP of the proposed animal feed are derived from methylotrophs. Methylotrophs are a diverse group of microorganisms that can use reduced one-carbon compounds, such as methanol or methane as the carbon source for their growth. While methylotrophs use such carbon sources, there are significant variations in their culture density, their productivity, and the properties of the resulting biomass. Optionally, the methylotrophs herein are fed methanol and not methane.

Bacterial strains from the Methylophilaceae family are found in fresh and marine water, soil, air and industrial wastewater treatment environments (Vorobev et al. 2013) and are comprised of four genera that include Methylophilus, Methylobacillus, Methylotenera and Methylovorus. Other genera of methylotrophs include Methylomonas, Methylobacter, Methylosinus, Methylocyctis, Methylomicrobium, Methylobacterium, Hyphomicrobium, Bacillus, Nocardia, Arthrobacter, Rhodopseudomonas, and Pseudomonas, Acidomonas, Methylococcus, Xanthobacter, Paracoccus, Arthrobacter, Rhodopseudomonas. These microorganisms are of interest for nutritional exploitation as sources of novel protein-rich feed ingredients as they are able to grow and utilize inexpensive single-carbon (C1) sources such as methanol (CH₃OH) or methane (CH₄) (Schrader et al. 2009; Ritala et al. 2017); made possible as strains within this family contain some key enzymes to breakdown such substrates (e.g., methanol dehydrogenase, methylamine dehydrogenase, methane monooxygenase) (Anthony 1982; Chistoserdova et al. 1991; Bodrossy and Kovacs 1994).

Optionally, the methylotrophic single cell protein products contain a methanol-fed methylotroph biomass, wherein the methylotrophs are not genetically modified (i.e., containing no inserted heterologous genes, artificially deleted sequences, or artificially altered sequences that increase or decrease the expression of a gene or genes). Unlike targeted genetic modifications, however, mutations occur spontaneously that can result in both genotypic and phenotypic changes, due to random chance or with selective pressure, such as laboratory passaging. The products of non-genetically modified organisms having only natural mutations (i.e. non-GMO) may be more readily accepted by consumers and regulatory bodies.

The standard reporting measure for protein content in food is crude protein. Because each amino acid contains nitrogen, crude protein is calculated by measuring the nitrogen content of a substance to give a rough estimate of protein content. Since not all nitrogen in food is in protein, crude protein can inflate the actual amount of total amino acids (i.e., true protein) in a food. True protein is calculated by directly measuring the amount of amino acid content, but is more time consuming and expensive than evaluating crude protein. True protein represents the total amino acids (methionine, arginine etc.) as a percentage of biomass. The true protein in a product has value, both monetarily and nutritionally.

For methylotrophic bacterial biomass products, crude protein is nearly always an overestimate, sometimes to a very large degree, and a high crude protein value therefore is not necessarily very meaningful. In two competing biomass products, the crude protein could be the same at 80%, but the amino acid content may be 60% in one product and 50% in the other.

Provided herein is a single cell protein product that optionally has a crude protein content of greater than or equal to 70%, 75%, 80%, or 85%. Optionally, the SCP product has a true amino acid or amino acid content that is greater than or equal to 45%, 50%, 55%, or 60% of the SCP product.

Optionally, the methylotrophic single cell protein product comprises all essential amino acids. Optionally, the apparent digestibility coefficient for each essential amino acid in the methylotrophic single cell protein product is at least 85% in Atlantic salmon. A variety of non-GMO methylotrophs include those from the genera Methylophilus, Methylobacillus, Methylotenera, Methylmonas, and Methylovorus. Optionally, the methylotroph is a Methylovorus menthalis (Strain J25) deposited with the International Depository of Canada (IDAC) under accession number 130619-01 on Jun. 13, 2019.

Optionally, the Essential Amino Acid Index in the single cell protein product is least 0.9, 0.91, 0.92, 0.93, 0.94, 0.95, 0.96, 0.97, 0.98, 0.99 or 1.0 for Atlantic salmon. The ten essential amino acids (leucine, isoleucine, valine, methionine, tryptophan, phenylalanine, threonine, arginine, lysine and histidine), are a vital requirement of an animal species. For a dietary regimen to be considered adequate for the support of all normal physiological functions, it should contain these essential amino acids in the appropriate levels and in the proper proportion of one to the other. Optionally, the single cell protein product includes 3-4% arginine, 1-2% histidine, 2-3.5% isoleucine, 3.5-6% leucine, 1.5-4.5% lysine, 1-2% methionine, 2-3% phenylalanine, 2.5-3.5% threonine, 0.01% to 1.5% tryptophan or 3-4.5% valine, or any percentage in between these recited percentages.

In addition to the essential amino acids, there are conditionally essential amino acids, meaning their synthesis can be limited under certain pathophysiological conditions, which in fish can be identified as cysteine, glutamine, hydroxyproline, proline and taurine. The function of non-essential amino acids is to provide a source of metabolizable nitrogen required by the animal organism for the biosynthesis of proteins, purines, nucleic acids, and other metabolites. Examples of non-essential amino acids include alanine, glycine, aspartic acid, and serine. Proper nutritional balance requires that these non-essential amino acids be provided in sufficient quantity and within appropriate relative proportions, although the range of proportions to each other that is less restrictive or critical than the balance required for the essential amino acids.

Apparent Digestibility Coefficient (ADC) is a measure of nutritive value and the ease of conversion of a digested product into useful nutrients by the digestive tract. Optionally, the apparent digestibility coefficient for each essential amino acid in the methylotrophic single cell protein product is at least 85%, 86%, 87%, 88%, 89%, 90%, 91%, 92%, 93%, 94%, 95%, 96%, 97%, 98%, 99% or 100% in Atlantic salmon. Thus, for each of leucine, isoleucine, valine, methionine, tryptophan, phenylalanine, threonine, arginine, lysine and histidine the apparent digestibility coefficient in the methylotrophic single cell protein product can be at least 85%, 86%, 87%, 88%, 89%, 90%, 91%, 92%, 93%, 94%, 95%, 96%, 97%, 98%, 99% or 100% in Atlantic salmon. Optionally, the digestibility coefficients in the methylotrophic single cell protein product can be at least 94%, 92%, 90%, 90%, 91%, 100%, 85%, 88%, 96%, and 90% for arginine, histidine, isoleucine, leucine, lysine, methionine, phenylalanine, threonine, tryptophan and valine, respectively.

Optionally, the methylotrophic single cell protein product has a high crude protein content. Because each amino acid contains nitrogen, crude protein is calculated by measuring the nitrogen content of a substance to give a rough estimate of protein content. Since not all nitrogen in food is in protein, crude protein can inflate the actual amount of true protein in a food. Crude protein is the total nitrogen in any food or feed product, multiplied by 6.25, a general factor that roughly corresponds to true protein in many foods and feeds. A crude protein of greater than 50% is expected for a bacterial product with a high true protein content. The methylotrophic single cell protein product provided herein comprises a crude protein content of greater than 50%, 60%, 70%, 80%, or 90%.

A lower relative carbohydrate content in a protein product is desirable as it indicates that the product will likely have a higher density of protein and lipids. Optionally, the methylotrophic single cell protein product comprising a carbohydrate content of 1% to 10%, 5% to 10%, 1% to 15%, 2% to 15%, 3%, to 15%, 4% to 15%, 5% to 15%, 6% to 15%, 7% to 15%, 8% to 15%, 9% to 15%, or 10% to 15%.

Optionally, the methylotrophic single cell protein product comprises 1% to 10% fatty acids. Optionally, 90% of the fatty acids are palmitic acid, palmitoleic acid and vaccenic acid. Optionally, the methylotrophic single cell protein comprises less than 1% of each of stearic acid, oleic acid and arachidonic acid.

Optionally, the methylotrophic SCP product comprises less than about 15% ash.

The SCP product described herein has a low odor. Without meaning to be limited by theory, the low odor may be due to the culturing method, use of methanol as the carbon source, or due to low content of certain components (e.g., ash). The limited odor may promote ingestion by the animal fed the SCP-containing feed, as a highly noxious odor could prove unpleasant to the animal.

Provided herein are methods of making the methylotrophic single cell protein product. Optionally, the method includes culturing a methylotroph under continuous fermentation conditions that promote growth of a biomass, wherein the culture conditions include a pH of 7 or less and a temperature of 33° C. or less; feeding the methylotroph culture methanol as a primary carbon source; and isolating the biomass of the culture to produce the methylotrophic single cell protein product.

Media for the process of producing biomass include, for example, a carbon source, a nitrogen source, salts, cofactors, buffers and other components required to grow and maintain the microorganism. If necessary, the media may also include certain substances required or beneficial for growth of the microorganism, for example vitamins, non-vitamin compounds, amino acids or nucleic acids. The process of producing biomass can be performed in various reactors, including continuous stirred tank bioreactors, bubble column bioreactors, fluidized bed bioreactors and packed bed bioreactors.

The process of producing biomass is performed using batch and continuous growth. In batch growth, all media components are added to the system and the bioreactor is inoculated. The fermentation proceeds without changes to the media, except for the addition of acid and/or base to maintain the pH, and air and/or oxygen to maintain dissolved oxygen levels. In continuous growth, defined media is added continuously and bioreactor contents removed continuously at the same rate.

To form a SCP product, the biomass is separated from the medium suitably by centrifugation, sedimentation, spray drying or filtration, or extracted and/or purified with a suitable solvent such as acetone, diethyl ether or chloroform. The protein product may comprise SCP that is heated, frozen, lyophilized or otherwise inactivated. Whole cells may be used as the final product or processed (e.g., lysed) to increase the bioavailability of the nutrients in the protein product.

In the provided methods, the primary or main carbon source comprises methanol. However, the carbon source may further include other sources of carbon, for example, carbohydrates, including sugars such as glucose, xylose, galactose, mannose, fructose, mannose and maltose; fats or oils such as corn oil or soybean oil; other alcohols such ethanol, propanol, butanol, pentanol or glycerol; and/or other organic molecules that the microorganism can use naturally or selected to use, alone or in a combination.

The nitrogen source may comprise an ammonium salt or nitrate salt, including but not limited to (NH₄)²SO₄, NH₃, NH₄Cl, (NH₄)²HPO₄, NH₄OH, KNO₃ or NaNO₃, alone or in a combination. Complex or organic nitrogen sources such as urea, yeast extract, casamino acids, peptone, tryptone, soy flour, corn steep liquor, or casein hydrolysate, alone or in a combination, may be used to supplement the media.

Salts may comprise H₃PO₄, KH₂PO₄, K₂HPO₄, MgSO₄, MgCl, ZnSO₄, MnSO₄, CaCl₂, CaCO₃, FeSO₄, KCl, CuSO₄, H₃BO₃, Na₂MoO₄, CoCl₂ and other salts alone or in combination.

Vitamins and/or non-vitamin compounds may comprise biotin, pantothenate, folic acid, inositol, nicotinic acid, p-aminobenzoic acid, pyridoxine, riboflavin, thiamine, cyanocobalamin, citric acid and ethylenediamine tetraacetic acid (EDTA).

As noted above, the methylotrophs are cultured under continuous fermentation conditions at a pH of 7 or less. Optionally, the pH of the media is between 1 to 7, 2 to 7, 3 to 7, 4 to 7, 5 to 7, or 6 to 7. The appropriate buffer to maintain or change the pH may be determined by a person skilled in the art.

The culturing temperature is a temperature of 33° C. or less. Optionally, the microorganism is cultured at a temperature of between 20° C. and 33° C. or between 24° C. and 33° C.

Also provided herein are animal feeds comprising the methylotrophic single cell protein product. Feeds are optionally produced by compression steam pelleting, cooking extrusion processing, or other conventional processes. In cooking extrusion, moistened SCP product is mixed, heated and sheared through an opening to expand or form cooked material. In pelleting, the SCP product is cooked with radiant heat or direct heat to a finished edible form. In various embodiments flavourings and/or colourings are added to the pellets.

Optionally, the animal feed comprises at least 5% methylotrophic single cell protein product and less than 20% plant-based protein products and less 35% animal-derived protein products, wherein the methylotrophic single cell protein product comprises a methanol fed methylotroph and wherein the methylotroph is not genetically modified. Optionally, the animal feed comprises at least 5% methylotrophic single cell protein and less than 40% plant-based protein products, wherein the methylotrophic single cell protein product comprises a methanol fed methylotroph of the genus methylovorus and wherein the methylotroph is not genetically modified. Optionally the animal feed also comprises less than 40% animal-derived protein. Optionally, the animal feed comprising a methylotrophic single cell protein product comprising all essential amino acids, wherein the methylotrophic single cell protein product comprises a methanol fed methylotroph that is not genetically modified, and wherein the apparent digestibility coefficient for each essential amino acid of the single cell protein product is at least 85% in Atlantic salmon.

The animal feeds provided herein can include at least 5%, 10%, 11%, 12%, 13%, 14%, 15%, 16%, 17%, 18%, 19%, 20%, 21%, 22%, 23%, 24%, 25%, 26%, 27%, 28%, 29% or 30% methylotrophic single cell protein. Optionally, the animal feed comprises 10% to 30% or 20% to 30% methylotrophic single cell protein product. Optionally, the methylotrophic single cell protein is the primary protein in the feed.

The animal feeds provided herein can include less than 30%, 25%, 20%, 15%, 10%, 5%, or 1% animal-derived protein products. Optionally, the animal feed comprises 1% to 20% fish meal. Optionally, the animal feed comprises no animal-derived protein products. Optionally, the animal feed contains no fish meal.

The animal feeds provided herein can include less than 40%, 35%, 30%, 25%, 20%, 15%, 10%, 5%, or 1% plant-based protein products. For example, the animal feeds can include 10% to 40%, 20% to 40%, 30% to 40%, 10% to 30%, or 20% to 30% plant-based protein products. Optionally, the animal feed comprises no plant-based protein products. Optionally, the animal feed does not comprise corn-based protein products. Optionally, the animal feed does not comprise soy-based protein products. Optionally, the animal feed does not contain corn-based and soy-based protein products.

Optionally, the animal feed contains no animal-derived protein products and no plant-based protein products.

Optionally, the animal feed comprises one or more of the following ingredients: vitamins (e.g., vitamin A, D, B₁₂, E, niacin, and betaine), synthetic amino acid additives (e.g., L-histidine, L-lysine, L-arginine, L-methionine), fats or oils, minerals (e.g., magnesium salts), probiotics, enzymes, flavors, preservatives, additives generally regarded as safe (GRAS) (e.g., acetic acid, sulfuric acid, aluminum salts, dextrans, glycerin, beeswax, sorbitol, and riboflavin).

Optionally, the animal feed is an aquafeed. Optionally, the aquafeed is feed for carnivorous fish.

Also provided herein are methods of feeding animals by feeding to the animal feed provided herein and described throughout the application. Optionally the animals are farmed fish, such carnivorous fish. Carnivorous fish include, for example, salmonids (e.g., salmon, including Atlantic salmon, trout and Arctic charr), cobia, carp, mahimahi, tuna, sea bass, sea bream, charr, catfish, tilapia, flounder, snapper, sturgeon, sole, cod and others. Optionally, the carnivorous fish can be a juvenile fish. As used herein, a juvenile fish refers to the stage of development between the post-larval stage of “fry” and the stage of the fish being commercially marketable but not yet sexually mature. The term “fry” refers to a fish in the stage after the larval state, when able to feed on exogenous food alone rather than endogenously provided by its larval stage yolk-sac. For example, the carnivorous fish can be a salmonid in the juvenile “pre-smolt” freshwater phase of their lifecycle. Alternatively, the carnivorous fish can be a salmonid in the “post-smolt” seawater phase of their lifecycle. During the “pre-smolt” phase, the salmonid is living in fresh water or fresh water conditions whereas a salmonid in “post-smolt” phase is living in sea or salt water conditions.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a composition or method is disclosed and discussed and a number of modifications that can be made to a number of molecules including the composition or method are discussed, each and every combination and permutation of the composition or method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

The examples below are intended to further illustrate certain aspects of the methods and compositions described herein, and are not intended to limit the scope of the claims.

### EXAMPLES

### Example 1—Biomass Production

Biomass was produced in a carbon limited, continuous culture process at a temperature of 33° C. The fermenter was agitated with a standard Ruston type impeller and aerated with air, such that the pO₂ was maintained at 20%. pH was maintained at 6.8 with a 4 N 1:1 ratio mixture of NaOH/KOH, and foaming was controlled with additions of commercially available antifoam.

To begin the process, seed cultures of the organism (Strain J25) were grown in shake flasks at 33° C. for approximately 18-24 hours, using either ATCC medium #1545, or pH adjusted medium of the type specified below, with a methanol concentration of 5 or 10 g/L. Seed culture was used to inoculate growth medium in the fermenter with the composition shown in Table 1 (per L).

TABLE 1

| Methanol | 10 | g |
|---|---|---|
| (NH₄)₂SO₄ | 5.4 | g |
| MgSO₄ 7H₂O | 0.67 | g |
| CaCl₂ | 10 | mg |
| FeSO₄ 7H₂O | 5 | mg |
| KCl | 14 | mg |
| H₃PO₄ | 0.93 mL (75%) | |
| CuSO₄ 5H₂0 | 0.1 | mg |
| H₃BO₃O | 0.07 | mg |
| MnSO₄ 1H₂O | 0.305 | mg |
| ZnSO₄ 7H₂0 | 0.5 | mg |
| Na₂MoO₄ 2H₂O | 0.12 | mg |
| CoCl₂ 6H₂0 | 0.1 | mg |

After batch growth concluded with medium containing 10 g/L methanol, continuous culture was then started using growth media containing 20 g/L methanol, of the same type specified above, with all other nutrient concentrations proportionally increased to match the carbon concentration. The dilution rate was increased from 0.05/h to 0.12/h over a 12 hour period and the culture was found to be methanol limited at that point. The dilution rate could then be gradually increased to 0.17/h.

### Example 2—Calculation of Yield

A sample of bacterial culture was collected by centrifugation at 10,000×g for 5 minutes, and then dried in an oven to produce the dried biomass for yield (g biomass/g methanol, expressed as a %) and productivity calculations. A productivity of at least 1.4 g/L/h was obtained using 20 g/L methanol (2%) and 8 g/L biomass.

### Example 3—Processing of Single Cell Protein

Biomass was collected in a refrigerated carboy during continuous fermentation, and centrifuged at ˜7,000×g before freezing (−40° C.). It was lyophilized and pulverized to a fine powder with a laboratory ultra-centrifugal mill.

### Example 4—Composition Analysis

The composition of J25 single cell protein product produced according to the process of Example 3 is provided in Table 2.

TABLE 2
Composition of J25 single cell protein
made according to process of Example 3.

| Characteristic | Value |
|---|---|
| Moisture | 3.8% |
| Ash | 8.4% |
| Crude Protein Content | 80-85% |
| Carbohydrate Content | 0.5% |
| True Amino Acid Content | 65% |
| Essential Amino Acid Content | 31% |
| Methionine Content | 1.8% |
| Methionine + Cysteine Content | 2.3% |
| Lysine Content | 3.9% |

### Example 5 —Methylophilus methylotrophus (ATCC 53528, NCIMB strain 10515, AS-1)

Methylophilus methylotrophus (ATCC 53528, NCIMB strain 10515, AS-1), commercialized by the Imperial Chemical Industries (Goldberg, 1986; MacLennan et al., 1974) was grown in shake flasks at 33° C. with 10 g/L methanol medium as described in Example 1, until mid-exponential growth phase. From flasks, 30% inoculum (v/v) was used to inoculate growth media of the same formulation, in a bioreactor under the conditions described in Example 1, for Methylovorus menthalis J25 (33° C., pO2 maintained at ≥20% with Rushton impellers and sparging air, pH maintained at 6.8 with 4N NaOH/KOH mixture, foam controlled with additions of antifoam). After batch phase ended, culture growth in 10 g/L methanol media occurred readily with dilution rates of 0.08/h to 0.15/h. Carbon limitation was confirmed at a 0.15/h dilution rate, using gas chromatography and the residual methanol level was as described in Vasey & Powell (1986). Biomass (g/L) was measured by oven drying centrifuged samples of culture. For 10 g/L methanol media and a dilution rate of 0.15/h, the yield was 3.94 g/L, or 39% (g biomass/g methanol), after 3 volume changes. The amino acid content of that sample was 53.5% and the amino acid profile is described in Table 3. The content of lysine in the biomass was 3.7% and the methionine content was 1.6%, lower than in Example 4. The essential amino acids were 26.5% of cell mass. Using 20 g/L methanol media with the formulation described in Example 4, and a dilution rate of 0.08/h, methanol limitation occurred within about 2 volume changes. However, when the dilution rate was set at 0.13/h and allowing 5 volume changes to reach steady state, the culture was not methanol limited, and the same was observed at 5.5 volume changes. The yield measured with 20 g/L methanol media and 0.13/h dilution rate was 24.4% (g biomass/g methanol).

TABLE 3
Amino acid profile of Methylophilus methylotrophus
(ATCC 53528)

| Characteristic | Value |
|---|---|
| True Amino Acid Content | 55.4% |
| Essential Amino Acid Content | 27.5% |
| Methionine Content | 1.7% |
| Methionine + Cysteine Content | 2.18% |
| Lysine Content | 3.9% |

Both organisms featured in Examples 4 and 5 are members of the Methylophilaceae family and metabolize methanol via the ribulose monophosphate (RuMP) pathway (Rosenberg et al., 2014). For both species, ammonium is assimilated to nitrogen via the glutamate cycle (Doronina et al., 2011; Rosenberg et al., 2014). M. methylotrophus is reported to produce relatively high concentrations of true protein (>50%) in continuous culture (Goldberg, 1986; Stringer, 1982), as was found for J25 (see Example 4). M. methylotrophus was also reported to produce as much as 4.8% lysine, and 2% methionine, a total 65% amino acid content, and with yields of >50%, after culture conditions had been optimized (Tannenbaum and Wang, 1975). Southgate and Goodwin (1989), cultured M. methylotrophus strain 10515 and found it was carbon limited with 15.3 g/L methanol media, and 0.13/h dilution rate, but the resulting yield (g biomass/g methanol) was less than 30%, similar to this example. MacLennan et al., (1974) reported a yield of 38% (g dried biomass/g methanol) for the same strain, with 56.4% amino acid content, including 4.6% lysine and 1.97% methionine.

There are metabolic similarities between the related species, both species can produce relatively high amino acid content and yield, and relatively high levels of lysine, methionine that can be produced. However, when M. methylotrophus was cultured under the conditions described (Example 5), J25 has a higher yield. The total amino acid content and content of essential amino acids in the J25 biomass was superior. J25 also reached a carbon limited state at a 0.17/h dilution with 20 g/L methanol, while M. methylotrophus did not reach a carbon limited state with the same methanol concentration at 0.13/h dilution rate and >5 volume changes. The combination of organism and process characteristics described in Examples 1-4, showed J25 had better results than M. methylotrophus by several metrics, which are important to process cost (e.g. yield), as well as total amino acid content, methionine, and essential amino acids, which are important factors for product quality.

TABLE 4
Comparison of amino acid profiles described in
Example 1, Example 5, and Gow et al., 1975, which discloses
the amino acid profile of Methylophilus methylotrophus
after optimizing growth conditions.

| | | Example 5 | Gow et al., 1975 |
|---|---|---|---|
| | Example 1 | M. | M. |
| Data Source | M. menthalis | methylotrophus | methylotrophus |
| Species/strain | J25 | (ATCC 53528) | (ATCC 53528) |
| Essential amino acid (EAA) | | | |
| Arginine (%) | 4.1 | 3.4 | 3.71 |
| Histidine (%) | 1.6 | 0.6 | 1.53 |
| Isoleucine (%) | 3.2 | 2.8 | 3.57 |
| Leucine (%) | 5.7 | 4.8 | 5.62 |
| Lysine (%) | 3.9 | 3.9 | 4.88 |
| Methionine (%) | 1.8 | 1.7 | 2.0 |
| Methionine + Cysteine (%) | 2.2 | 2.18 | 2.5 |
| Phenylalanine (%) | 2.8 | 2.64 | 2.85 |
| Phenylalanine + Tyrosine (%) | 5.7 | 5.2 | 5.4 |
| Threonine (%) | 3.5 | 2.8 | 3.81 |
| Tryptophan (%) | 0.1 | 1.3 | 0.74 |
| Valine (%) | 4.4 | 3.6 | 4.34 |
| Σ EAA (%) | 31.1 | 27.5 | 33.1 |
| Non-essential amino acid (NEAA) | | | |
| Alanine (%) | 4.9 | 4.1 | 5.66 |
| Aspartic acid (%) | 7.3 | 6.2 | 7.08 |
| Cysteine (%) | 0.4 | 0.4 | 0.51 |
| Glutamic acid (%) | 8.3 | 6.9 | 7.97 |
| Glycine (%) | 3.8 | 3.2 | 4.18 |
| Proline (%) | 2.7 | 2.2 | 2.5 |
| Serine (%) | 2.8 | 2.3 | 2.82 |
| Taurine (%) | | | |
| Tyrosine (%) | 2.9 | 2.6 | 2.55 |
| Σ NEAA (%) | 33.1 | 27.9 | 33.3 |
| EAA:NEAA | 0.94 | 0.99 | 0.99 |
| total AA (%) | 64.2 | 55.4 | 66.3 |

### Example 6—Apparent Digestibility of Proximate Nutrients, Energy and Essential Amino Acids of a Novel Methylotrophic Single-Cell Protein (SCP) Meal and its Effect on Diet Digestibility at Partial or Complete Replacement of Major Plant-Proteins for Atlantic Salmon, Salmo salar

This example establishes the Atlantic salmon-specific apparent digestibility coefficients (ADCs) of the proximate nutrients, energy and Essential Amino Acids (EAAs) in the Methylovorus menthalis (strain J25) single cell protein (SCP) meal using the substitution digestion assay (NRC 2011). This example also determines the effect of partial (50%) or complete replacement (100%) of these major plant-protein ingredients with J25 SCP meal on in vivo feed intake and digestibility of dietary proximate nutrients and EAAs.

### Materials and Methods

### Single-Cell Protein (SCP) Meal

The SCP meal used in this study was produced from a strain of the methylotrophic bacteria Methylovorus menthalis (strain J25). A Biostat B fermenter (Sartorius Stedim, Germany), equipped with a 5 L vessel was used to produce the SCP at 33° C. Fermentation pH was maintained at 6.8, using 4 N 108 NaOH/KOH mixture. Air was delivered with a ring sparger at 1-4 VVM, and agitation with Rushton impellers 109 maintained pO2 at ≥20%. Batch growth was initiated in growth media containing 10 g L-1 methanol, with the addition of 20-30% of the working volume of culture from shake flasks. At the end of batch growth, pO2 rapidly increased, indicating methanol was exhausted from the system. Continuous culture began with the addition of 20 g L-1 methanol media at a dilution rate of approximately 0.05-0.1 h-1. The dilution rate was gradually increased to approximately 0.15 h-1. The filter-sterilized growth media (per L) consisted of: (NH₄)²SO₄, 10.8 g; MgSO₄·7H₂O, 1.34 g; CaCl₂, 20 mg; FeSO₄·7H₂O, 10 mg; KCl, 28 mg; H₃PO₄ (75%), 1.86 mL; CuSO₄·5H₂O, 0.2 mg; H₃BO₃, 0.14 mg; MnSO₄·1H₂O, 0.61 mg; ZnSO₄·7H₂O, 1 mg; Na₂MoO₄.2H₂O, 0.24 mg; CoCl₂·6H₂O, 0.2 mg and CH₃OH (methanol), 20 g. Growth media with 10 g L-1 methanol was produced by reducing the concentration of all the above components by half. After allowing at least 3 volume changes to elapse, methanol limitation in the steady-state culture was verified by gas chromatography (NCASI method DI/MEOH-94.03). At that point, culture was collected continuously in a carboy and refrigerated)(4° C. Chilled culture was centrifuged (8,000× g at 4° C. for 20 minutes) within 24 hours of collection (Sorvall RC5C+ centrifuge) and contained approximately 8 g biomass L-1 (dry weight basis). Wet cell pellets (paste) were continually pooled and stored at −40° C. until the total biomass produced was sufficient for lyophilization. A total of three production campaigns generated approximately 12.5 kg of frozen paste (˜20% solids) for this study and was lyophilized for 96 h at a low shelf temperature (<5° C.) in a large capacity freeze-dryer (model 35EL, The Virtis Company, Gardiner, NY) to a final moisture content of <4%. Freeze-dried J25 SCP biomass was pulverized to pass through a 500 μm screen at 10,000 rpm using a laboratory ultra-centrifugal mill (model ZM200, Retsch GmbH., Haan, Germany) equipped with a Retsch pneumatic auto-feeder (model DR100). This resulted in ˜2.5 kg of freeze-dried, powdered J25 SCP meal for this study which was kept frozen at −80° C. until use.

### Experimental Test Diets.

A practical-ingredient control diet (free of J25 SCP meal) was formulated to meet the known dietary requirements of juvenile Atlantic salmon reared in freshwater (NRC 2011). This diet was formulated to closely resemble a commercial pre-smolt feed equivalent benchmark typically used in the Canadian salmon farming industry. Following the substitution digestion assay (NRC 2011), an aliquot of 80% control diet was blended (% w:w basis) with 20% J25 SCP meal to form the test diet (Table 5).

TABLE 5
Formulation of the experimental test diets used to measure in vivo
apparent digestibility of the J25 SCP meal fed to juvenile Atlantic salmon
(Salmo salar) following the substitution digestion assay (as-is basis).

| | % of diet | % of diet |
|---|---|---|
| | Control Diet | Substitution Diet |
| Ingredient | (J25 SCP meal-free) | (80:20) |
| Fish meal (71% Crude Protein (CP)) | 18.260 | 14.608 |
| Soy protein concentrate (63% CP) | 22.730 | 18.184 |
| Corn protein concentrate (78% CP) | 8.000 | 6.400 |
| J25SCP meal (81% CP) | — | 20.000 |
| Poultry by-product meal (71% CP) | 7.5000 | 6.000 |
| Wheat gluten meal (81% CP) | 5.000 | 4.000 |
| Blood meal (91% CP) | 5.000 | 4.000 |
| Whole wheat flour | 11.6000 | 9.280 |
| Corn starch, dextrimized | 0.590 | 0.472 |
| Fish oil | 8.140 | 6.512 |
| Poultry fat | 4.070 | 3.256 |
| Canola oil | 4.070 | 3.256 |
| Vitamin and Mineral mixture^{a} | 0.400 | 0.320 |
| Calcium phosphate, monobasic | 2.900 | 2.320 |
| Choline chloride | 0.400 | 0.320 |
| Vitamin C, ascorbic acid ‘Stay-C 35’ | 0.030 | 0.24 |
| Vitamin E, α-tocopherol | 0.030 | 0.24 |
| Salt, NaCl | 0.250 | 0.200 |
| L-lysine | 0.350 | 0.280 |
| L-methionine | 0.180 | 0.144 |
| Chromic oxide | 0.500 | 0.400 |
| Total | 100.000 | 100.000 |

^{a}Freshwater salmonid mixture (Corey Nutrition, Fredericton, NB, Canada)

As a means to evaluate the effect of partial or complete replacement of dietary plant-based ingredients (e.g., soy and corn protein) with J25 SCP meal on nutrient digestibility, two nutritionally-balanced test diets were formulated to be isonitrogenous (50% crude protein), isolipidic (22% fat) and isocaloric (19 MJ kg-1 digestible energy); representing 50 and 100% replacement levels (Table 6).

TABLE 6
Formulation of the nutritionally-balanced experimental test diets used to measure
the effect of J25 SCP meal replacement of dietary plant-based ingredients on in vivo
apparent digestibility of Atlantic salmon (Salmo solar) feeds (as-is basis).

| | | % of diet | % of diet |
|---|---|---|---|
| | % of diet | Plant protein | Plant protein |
| | Control diet | replacement diet | replacement diet |
| Ingredient | (J25 SCP meal-free) | (50% replacement) | (100% replacement) |
| Fish meal (71% CP) | 18.260 | 18.260 | 18.260 |
| Soy protein concentrate (63% CP) | 22.730 | 11.365 | — |
| Corn protein concentrate (78% CP) | 8.000 | 4.000 | — |
| J25 SCP meal (81%) | — | 13.000 | 25.600 |
| Poultry by-product meal (71% CP) | 7.500 | 7.500 | 7.500 |
| Wheat gluten meal (81% CP) | 5.000 | 5.000 | 5.000 |
| Blood meal (91% CP) | 5.000 | 5.000 | 5.000 |
| Whole wheat flour | 11.600 | 11.600 | 11.600 |
| Corn starch, dextrimized | 0.590 | 4.135 | 9.200 |
| Fish oil | 8.140 | 8.000 | 7.500 |
| Poultry fat | 4.070 | 4.000 | 3.750 |
| Canola oil | 4.070 | 4.000 | 3.750 |
| Vitamin and Mineral mixture^{a} | 0.400 | 0.400 | 0.400 |
| Calcium phosphate, monobasic | 2.900 | 2.000 | 0.700 |
| Choline chloride | 0.400 | 0.400 | 0.400 |
| Vitamin C, ascorbic acid ‘Stay-C 35’ | 0.030 | 0.030 | 0.030 |
| Vitamin E, α-tocopherol | 0.030 | 0.030 | 0.030 |
| Salt, NaCl | 0.250 | 0.250 | 0.250 |
| L-lysine | 0.350 | 0.350 | 0.350 |
| L-methionine | 0.180 | 0.180 | 0.180 |
| Chromic oxide | 0.500 | 0.500 | 0.500 |
| Total | 100.000 | 100.000 | 100.000 |

^{a}Freshwater salmonid mixture (Corey Nutrition, Fredericton, NB, Canada)

All test diets were supplemented with chromic oxide (Cr2O3, 0.5% w:w basis) as the inert digestion indicator. Dry ingredients were finely ground (≤500 μm) using the same laboratory ultra-centrifugal mill described previously. Micronutrients (e.g., vitamins, minerals, amino acids) were pre-mixed with whole wheat flour using a Globe® benchtop planetary mixer (model SP-20, Globe Food Equipment Company, Dayton, OH) prior to addition to the main ingredient mixture. All ingredients were thoroughly blended in a Hobart® floor planetary mixer (model H600T, Rapids Machinery Corporation, Troy, OH) and compression steam pelleted into 2.5 mm pellets (model CL-2, California Pellet Mill Co., San Francisco, CA). The pellets were forced-air dried at 80° C. for 90 minutes to form dry, sinking pellets and stored in air-tight containers at −20° C. until use. Diets were screened to remove fines prior to feeding.

### Digestibility Assay

Apparent digestibility coefficients (ADCs) of dry matter, protein, energy and essential amino acids for the test diets and the J25 SCP meal were measured using the indirect digestibility determination method (NRC 2011). Specially-designed tanks as described in Tibbetts et al. Aquaculture 261, 1314-1327 (2006) were used for passive collection of naturally egested fecal material from fish voluntarily consuming the various test diets. Digestibility measurements were made using 476 juvenile Atlantic salmon (average weight; 23.7±1.0 g fish-1) obtained from a local hatchery (Marine Harvest Fish Hatchery, Cardigan, PE, Canada). The fish were acclimated to the experimental conditions for a 14-day period while being gradually weaned from a commercial diet onto their respective test diets. During this time, they were hand-fed to apparent voluntary satiety four times daily (08:00, 11:00, 13:00 and 15:00 h). The commercial diet (3.0 mm extruded salmonid feed, EWOS/Cargill Canada, Surrey, BC, Canada) contained 6% moisture, 50% crude protein, 19% lipid, 11% ash and 23 MJ kg-1 gross energy (as-fed basis). The fecal collection period lasted until a minimum of 50 g of wet fecal material was collected from each tank (11 days) and each of 4 test diets was fed to triplicate tanks (initial stocking density, 9.4±0.6 kg m-3). De-gassed and oxygenated freshwater from a well was supplied to each tank at a flow rate of 5 L min-1 in a flow-through system and water temperatures and dissolved oxygen levels were recorded daily (14.0±0.2° C. and 10.8±0.5 mg L-1, respectively). During the experimental period, fish were hand-fed to apparent voluntary satiety four times daily (08:00, 11:00, 13:00 and 15:00 h). The tanks were checked daily for dead or moribund fish and none were found throughout the study. Each day, after the final feeding, the tanks and fecal collection columns were thoroughly cleaned with a brush to remove residual particulate matter (feces and uneaten feed) and rinsed thoroughly with freshwater. Fecal samples were collected each morning (08:00 h) into 50 mL plastic conical bottom tubes, centrifuged (4,000 rpm [2560×g] for 20 min at 4° C.) and the supernatant carefully decanted and discarded and each sample stored in a sealed container at −20° C. for the duration of the collection period. Fecal samples were lyophilized for 72 h at a low shelf temperature (≤5° C.) to a final moisture content of <3%. The study was conducted in compliance with guidelines set out by the Canadian Council on Animal Care (CCAC 2005).

### Analytical Techniques

Single-cell protein (SCP) meal, test diets and lyophilized fecal samples were analyzed using similar procedures. Moisture and ash contents were determined gravimetrically by drying in an oven at 105° C. and by incineration in a muffle furnace at 550° C. for 18 h. Nitrogen (N) contents were determined by elemental analysis (950° C. furnace) using a Leco N analyzer (model FP-528, Leco Corporation, St. Joseph, MI) with ultra-high purity oxygen as the combustion gas and ultra-high purity helium as the carrier gas and crude protein content calculated as N×6.25. Crude lipids were extracted by solvent extraction on a Soxtec™ automated system (model 2050, FOSS North America, Eden Prairie, MN, USA) using HPLC-grade chloroform:methanol (2:1 v:v) at 150° C. for 82 minutes. Carbohydrate contents were estimated as 100%−(crude protein+crude lipid+ash). Starch contents were determined by the α-amylase and amyloglucosidase method (AOAC Official Method 996.11 and AACC Method 76.13) using a Total Starch Assay Kit (K-TSTA, Megazyme International Ireland Ltd., Ireland). Crude fiber contents were estimated using the ANKOM filter bag technique (AOCS 2005). Gross energy (MJ kg-1) contents were measured using an isoperibol oxygen bomb calorimeter (model 6200, Parr Instrument Company, Moline, IL) equipped with a Parr 6510 water handling system for closed-loop operation. Elemental compositions were measured by ICP-AES according to SW-846 Method 6010C and mercury was measured following reference method 7471B (EPA 2007). Concentrations of minerals, trace elements and heavy metals were determined using element-specific wavelengths on an IRIS Intrepid II spectrometer (Thermo Fisher Scientific, Waltham, MA). Lipid fractions were extracted by methanolic HCl in-situ transesterification (McGinn et al. Algal Res. 1, 155-165 2012) and the corresponding fatty acid methyl esters (FAMEs) were separated and quantified by GC-FID (Omegawax 250 column, Agilent 7890). Individual FAs, along with an internal standard (C19:0; methyl nonadecanoate, Fluka), were identified by comparing retention times to two FA reference mixtures (Supelco 37 and PUFA No. 3, Sigma-Aldrich). Chromic oxide concentrations of test diets and lyophilized fecal samples were determined by flame atomic absorption spectrophotometry (model iCE 3000 Series AA, Thermo Fisher Scientific, Waltham, MA) following phosphoric acid and potassium bromide digestion (Williams et al. J. Agric. Sci. 59, 381-385 1962). Amino acid concentrations were determined using the Waters Pico-Tag RP-HPLC method (Heinriksen and Meredith Anal. Biochem. 136, 65-74, 1984; White et al. J. Clin. Lab. Auto. 8, 170-177, 1986). Essential amino acid index (EAAI) was calculated according to Oser (J. Am. Diet. Assoc. 27, 396-402 1951) relative to an ideal protein pattern (egg albumin) and protein digestibility-corrected amino acid score (PDCAAS) and digestible indispensable amino acid score (DIAAS) were calculated according to Schaafsma J. Nutr. 130, 1865-1867 (2000) and Rutherfurd et al. J. Nutr. 145, 372-379 (2015) relative to the known dietary requirements of pre-smolt, freshwater-phase juvenile Atlantic salmon (NRC 2011). All analytical work was conducted in triplicate.

### Calculations and Statistical Methods

In vivo ADCs of dry matter (DM), protein (P), energy (E) and essential amino acids (EAA) of the diets were calculated on a dry-weight basis according to the following equations (NRC 2011):

ADC of DM (%)=100-100×Chromic oxide in test diet/(%)Chromic oxide in feces (%)

ADC of P, E or EAA (%)=100-100×Chromic oxide in test diet/(%) Chromic oxide in feces (%)×P, E or EAA in feces (% or cal per g)/P, E or EAA in test diet (% or cal per g) 211

Using these data, in vivo ADCs for the single J25 SCP meal were calculated on a dry-weight basis according to NRC (2011):

J25 SCP meal ADC (%)=ADC of test diet+(ADC of test diet−ADC of control diet)×ρcontrol diet×D control diet/ρJ25 SCP meal×D J25 SCP meal
Where ‘ρ’ represents the proportion of the control diet or J25 SCP meal in the combined test diet and ‘D’ represents the DM, P, E or EAA content of the control diet or J25 SCP meal.

Data are reported as mean±standard deviation. Statistical analyses were performed using one-way analysis of variance, ANOVA (SigmaStat® v.3.5 (Systat Software, Inc.)) with a 5% level of probability (P<0.05) selected in advance to sufficiently demonstrate a statistically significant difference. Where significant differences were observed, treatment means were differentiated using pairwise comparisons using the Tukey test. Raw data was checked for normality using the Kolmogorov-Smirnov test (SigmaStat® v.3.5).

### Results

### Composition of J25 SCP Meal

Proximate and amino acid composition of the J25 SCP meal used in this example are shown in Table 7 and its EAA profile, PDCAAS and DIAAS values are shown in Table 8.

TABLE 7
Proximate and amino acid (AA) composition of the J25 SCP meal.

| Proximate Composition | |
|---|---|
| Moisture (%) | 3.8 |
| Ash (%) | 8.4 |
| Crude protein (%) | 81.4 |
| Crude lipid (%) | 9.7 |
| Carbohydrate (%) | 0.5 |
| Starch (%) | 0.8 |
| Crude fiber (%) | <0.1 |
| Gross energy (MJ kg−1) | 21.3 |
| Essential Amino Acid (EAA) | |
| Arginine (%) | 4.1 [1.5-2.2]¹ {2.3}² |
| Histidine (%) | 1.6 [0.7-0.8]¹ {2.0}² |
| Isoleucine (%) | 3.2 [1.0-1.1]¹ {2.9}² |
| Leucine (%) | 5.7 [1.5-1.6]¹ {3.8}² |
| Lysine (%) | 3.9 [2.2-2.4]¹ {1.6}² |
| Methionine (%) | 1.8 [0.7]¹ {2.6}² |
| Methionine + Cysteine (%) | 2.3 [1.1]¹ {2.1}² |
| Phenylalanine (%) | 2.8 [0.9]¹ {3.1}² |
| Phenylalanine + Tyrosine (%) | 5.7 [1.8]¹ {3.2}² |
| Threonine (%) | 3.5 [1.1]¹ {3.2}² |
| Tryptophan (%) | <0.1 [0.3]¹ {0.3}² |
| Valine (%) | 4.4 [1.2]¹ {3.7}² |
| Non-essential amino acid (NEAA) | |
| Alanine (%) | 4.9 |
| a-amino-N-butyric acid (%) | 0.8 |
| Aspartic acid (%) | 7.3 |
| Cysteine (%) | 0.4 |
| Glutamic acid (%) | 8.3 |
| Glycine (%) | 3.8 |
| Ornithine (%) | <0.1 |
| Proline (%) | 2.7 |
| Serine (%) | 2.8 |
| Taurine (%) | <0.1 |
| Tyrosine (%) | 2.9 |
| Σ AA (%) | 65.2 |
| Σ EAA (%) | 31.1 |
| Σ NEAA (%) | 34.1 |
| EAA:NEAA ratio | 0.9 |

¹Values in [brackets] are the ‘dietary requirements’ for each essential amino acid relative to dietary requirements of Atlantic salmon, Pacific salmon and rainbow trout (NRC 2011).
²Values in {parenthesis} are the ‘chemical score’ for each essential amino acid relative to dietary requirements of Atlantic salmon (NRC 2011).

TABLE 8
Essential amino acid (EAA) profile, protein digestibility-corrected
amino acid score (PDCAAS) and digestible indispensable amino
acid score (DIAAS) of the J25 SCP meal (DW basis).

| | EAA profile (g 100 g | | |
|---|---|---|---|
| EAA | protein−1) | PDCAAS | DIAAS |
| Arginine | 6.3 | 2.1 | 2.2 |
| Histidine | 2.5 | 1.8 | 1.9 |
| Isoleucine | 4.9 | 2.7 | 2.6 |
| Leucine | 8.7 | 3.5 | 3.4 |
| Lysine | 5.9 | 1.5 | 1.5 |
| Methionine | 2.8 | 2.4 | 2.6 |
| Phenylalanine | 4.2 | 2.9 | 2.7 |
| Threonine | 5.4 | 2.9 | 2.8 |
| Tryptophan | <0.1 | 0.3 | 0.3 |
| Valine | 6.8 | 3.4 | 3.3 |
| EAA index | 0.9 | | |

The J25 SCP meal was rich in crude protein (81%) and total amino acids (65%) and was energy-dense (21 MJ kg-1 gross energy) with a moderate amount of crude lipid (10%) and low in moisture (4%), ash (8%) and carbohydrates like starch and crude fibre (<1%). The essential (EAA) and non-essential amino acid (NEAA) content of the J25 SCP meal is well-balanced with an EAA:NEAA ratio of 0.9. The composition of EAAs (expressed as % of sample on an as-is basis) in the J25 SCP meal is leucine (6%)>valine (4%)>arginine (4%)>lysine (4%)>10 threonine (4%)>isoleucine (3%)>phenylalanine (3%)>methionine (2%)>histidine (2%)>tryptophan (<1%). The composition of NEAAs is glutamic acid (8%)>aspartic acid (7%)>alanine (5%)>glycine (4%)>tyrosine (3%)>serine (3%)>proline (3%)>α-amino-N-butyric acid (1%) with trace concentrations (<1%) of cysteine, ornithine and taurine. The EAA profile (expressed as g 100 g protein-1 on a DW basis) is leucine (9%)>valine (7%)>arginine (6%)>lysine (6%)>threonine (5%)>isoleucine (5%)>phenylalanine (4%)>methionine (3%)>histidine (3%)>tryptophan (<1%); with a high EAA index (0.9). The EAAs of the J25 SCP meal (excluding tryptophan) show exceptionally high PDCAAS and DIAAS values for leucine (3.4-3.5)>valine (3.3-3.4)>threonine (2.8-2.9)>phenylalanine (2.7-2.9)>isoleucine (2.6-2.7)>methionine (2.4-2.6)>arginine (2.1-2.2)>histidine (1.8-1.9)>lysine (1.5)>tryptophan (0.3). As demonstrated, the J25 SCP meal used in this study is predominantly composed of protein, however it also contains a moderate amount of lipid (˜10%). The lipid fraction of J25 SCP meal has a very simple profile, composed almost entirely (˜90% of total FA) of saturated fatty acid C16:0 palmitic acid (46% of total FA or 4% of sample) and monounsaturated fatty acid C16:1n-7 palmitoleic acid (44% of total FA or 3% of sample). For general interest, the elemental concentrations, including minerals, trace elements and heavy metals, of the J25 SCP meal are provided in Table 9.

TABLE 9
Elemental concentrations of the J25 SCP meal.

| | |
|---|---|
| Minerals (%) | |
| Calcium | 0.05 |
| Magnesium | 0.49 |
| Phosphorous | 2.83 |
| Potassium | 0.77 |
| Sodium | 0.23 |
| Ca:P raio | 0.02 |
| Trace elements (mg kg−1) | |
| Aluminum | 99.7 |
| Barium | 1.0 |
| Chromium | 0.2 |
| Cobalt | 4.0 |
| Copper | 14.0 |
| Iron | 463.3 |
| Manganese | 14.0 |
| Molybdenum | 9.5 |
| Nickel | 1.2 |
| Strontium | 0.3 |
| Titanium | 0.9 |
| Vanadium | 5.1 |
| Zinc | 60.0 |
| Zirconium | 2.6 |
| Heavy metals^{a} (ppm) | |
| Aluminum | 99.7 |
| Arsenic | 0.4 |
| Cadmium | 0.2 |
| Lead | <DL^{b} |
| Mercury | <DL^{b} |

^{a}Maximum acceptable levels (ppm) according to the Canadian Food Inspection Agency = Aluminum (200), Arsenic (8), Cadmium (0.4), Lead (8), Mercury (0.1-0.5).
^{b}Below detection limit.

### Composition of the Experimental Test Diets

Proximate and amino acid composition of the experimental test diets are shown in Table 10.

TABLE 10
Proximate and amino acid (AA) composition of the
experimental test diets used to evaluate in vivo
apparent 1123 digestibility of J25 SCP meal.

| | Control | Substi- | Plant protein | Plant protein |
|---|---|---|---|---|
| | diet | tution | replacement | replacement |
| | (J25 SCP | diet | diet (50% | diet (100% |
| | free-meal) | (80:20) | replacement) | replacement) |
| Moisture (%) | 6.2 | 5.7 | 5.9 | 5.4 |
| Ash (%) | 8.4 | 8.0 | 7.8 | 7.2 |
| Crude protein | 49.2 | 56.4 | 49.5 | 50.5 |
| (%) | | | | |
| Crude lipid (%) | 22.3 | 20.4 | 22.3 | 21.7 |
| Carbohydrate | 20.1 | 15.2 | 20.4 | 20.6 |
| (%) | | | | |
| Gross energy | 22.3 | 22.1 | 22.6 | 22.5 |
| (MJ kg−1) | | | | |
| Essential amino | | | | |
| acid (EAA) | | | | |
| Arginine (%) | 2.5 | 2.6 | 2.4 | 2.6 |
| Histidine (%) | 0.5 | 0.4 | 0.4 | 0.6 |
| Isoleucine (%) | 1.4 | 1.6 | 1.5 | 1.4 |
| Leucine (%) | 3.6 | 3.6 | 3.3 | 3.0 |
| Lysine (%) | 2.3 | 2.5 | 2.4 | 2.1 |
| Methionine (%) | 0.9 | 0.9 | 1.0 | 1.1 |
| Phenylalanine | 1.9 | 1.9 | 1.7 | 1.5 |
| (%) | | | | |
| Threonine (%) | 1.3 | 1.5 | 1.4 | 1.7 |
| Tryptophan (%) | 0.3 | 0.3 | 0.3 | 0.2 |
| Valine (%) | 2.3 | 2.5 | 2.4 | 2.6 |
| Nonessential | | | | |
| amino acids | | | | |
| (NEAA) | | | | |
| Alanine (%) | 2.1 | 2.4 | 2.3 | 2.7 |
| Aspartic acid (%) | 3.8 | 4.1 | 3.8 | 4.3 |
| Cysteine (%) | 0.4 | 0.4 | 0.3 | 0.3 |
| Glutamic acid | 7.8 | 7.2 | 6.7 | 7.0 |
| (%) | | | | |
| Glycine (%) | 1.7 | 1.9 | 1.9 | 2.4 |
| Hydroxyproline | 0.2 | 0.1 | 0.2 | 0.2 |
| (%) | | | | |
| Proline (%) | 2.4 | 2.2 | 2.1 | 2.2 |
| Serine (%) | 1.8 | 1.9 | 1.7 | 1.8 |
| Taurine (%) | 0.1 | 0.1 | 0.1 | 0.1 |
| Tyrosine (%) | 1.5 | 1.6 | 1.5 | 1.5 |

The test diets used in the substitution digestion assay had similar levels of moisture (6%), ash (8%) and gross energy (22 MJ 252 kg-1) and variable levels of crude protein (49-56%), crude lipid (20-22%) and carbohydrate (15-20%). Essential and non-essential amino acid compositions were similar between the two diets. As formulated, the test diets used in the nutritionally-balanced digestion assay had highly similar levels of moisture (5-6%), ash (7-8%), crude 255 protein (49-50%), crude lipid (22%), carbohydrate (20-21%) and energy (22-23 MJ kg-1) with highly similar concentrations of EAAs and non-EAAs.

### Diet Palatability and Fish Performance

No mortalities occurred over the course of the feeding trials for fish consuming any of the experimental test diets. Throughout the trial, the test feeds were fed to each tank of fish four times daily to apparent satiety and they were consumed at a statistically equal rate (0.45±0.03 g feed fish-1 day-1; P=0.283) or ˜2% of their BW per day. This suggests that J25 SCP meal at the dietary inclusion levels investigated (13-26%) caused no positive or negative chemosensory effects in the test feeds; relative to the practical-ingredient control diet (J25 SCP meal-free) which was representative of industrial juvenile farmed Atlantic salmon feeds used in Canada. In addition, while the present study was not an exhaustive growth performance trial per se; fish fed diets supplemented with the J25 SCP meal showed statistically the same final body weight (31.5±1.4 g fish-1; P=0.163), weight gain (7.8±0.9 g fish-1; P=0.649), thermal growth coefficient (0.12±0.01 g⅓ degree day-1; P=0.807) and feed conversion ratio (0.90±0.07 g feed g gain-1; P=0.896). Due to the relatively short duration of in vivo digestibility assays, longer-term growth performance and fish health studies are presently underway to scrutinize these encouraging findings.

### Dry Matter, Protein, Energy and Essential Amino Acid Digestibility: Substitution Digestion Assay

Apparent digestibility coefficients (ADCs) of dry matter, protein, energy and essential amino acids for the test diets used in the substitution digestion assay and the J25 SCP meal itself are shown in Table 11.

TABLE 11
Apparent digestibility coefficients (in vivo ADCs, %) of dry matter,
protein, energy and essential amino acids (EAAs) in the experimental
test diets and the J25 SCP meal following the substitution digestion
assay with juvenile Atlantic salmon (Salmo salar).

| | Control diet | Substitution | Single- |
|---|---|---|---|
| | (J25 SCP | diet | ingredient |
| | free-meal) | (80:20) | J25 SCP meal |
| Proximate nutrient | | | |
| Dry Matter (%) | 75.4 ± 0.4^{a} | 77.5 ± 0.4^{b} | 85.3 ± 1.7 |
| Protein (%) | 93.1 ± 0.2^{ns} | 92.9 ± 0.2 | 92.4 ± 0.6 |
| Energy (%) | 84.0 ± 0.4^{ns} | 84.7 ± 0.2^{a} | 87.9 ± 1.1 |
| Essential amino acid | | | |
| (EAA) | | | |
| Arginine (%) | 96.0 ± 0.3^{ns} | 95.6 ± 0.3 | 94.5 ± 1.2 |
| Histidine (%) | 95.8 ± 0.3^{ns} | 94.3 ± 1.5 | 92.6 ± 3.1 |
| Isoleucine (%) | 92.9 ± 0.3^{ns} | 92.1 ± 0.5 | 90.5 ± 1.6 |
| Leucine (%) | 94.8 ± 0.2^{a} | 93.6 ± 0.3^{b} | 90.6 ± 1.1 |
| Lysine (%) | 96.3 ± 0.2^{a} | 94.9 ± 0.4^{b} | 91.0 ± 1.6 |
| Methionine (%) | 94.5 ± 0.3^{ns} | 97.3 ± 2.7 | 95.7 ± 2.2 |
| Phenylalanine (%) | 93.3 ± 0.3^{a} | 91.3 ± 0.4^{b} | 85.5 ± 1.7 |
| Threonine (%) | 93.9 ± 0.5^{a} | 91.9 ± 0.7^{b} | 88.5 ± 2.0 |
| Tryptophan (%) | 99.5 ± 0.2^{ns} | 99.4 ± 0.1 | 96.3 ± 6.0 |
| Valine (%) | 94.4 ± 0.3^{a} | 93.3 ± 0.4^{b} | 90.9 ± 1.3 |

^{a}Values within the same row (diets only) having different superscript letters are significantly different (P < 0.05).

The test diet containing 20% J25 SCP meal was digested at statistically the same levels as the control diet (J25 SCP meal-free) for protein (93%; P=0.369), arginine (96%; P=0.267), histidine (94-96%; P=0.214), isoleucine (92-93%; P=0.129), methionine (95-97%; P=0.138) and tryptophan (99%; P=0.514), while its dry matter ADC (78%) and energy ADC (85%) were significantly higher (P<0.035) than those of the control diet (75 and 84%, respectively). The diet containing 20% J25 SCP meal was digested at slightly lower levels than the control diet for leucine (94 vs. 95%; P=0.004), lysine (95 vs. 96%; P=0.005), phenylalanine (91 vs. 93%; P=0.001), threonine (92 vs. 94%; P=0.012) and valine (93 vs. 94%; P=0.017). Nutrient ADC values for the J25 SCP meal itself were high for dry matter (85%), protein (92%), energy (88%) and all EAAs (average, 92%; range, 86-96%).

### Plant-Protein Replacement Assay

Apparent digestibility coefficients (ADCs) of dry matter, protein, energy and essential amino acids in the test diets used in the plant-protein replacement digestion assay are shown in Table 12.

TABLE 12
Apparent digestibility coefficients¹ (in vivo ADCs, %)
of the nutritionally-balanced experimental test diets
used to measure the effect of J25 SCP meal replacement of
dietary plant-based ingredients on in vivo apparent digestibility
of Atlantic salmon (Salmo salar) feeds.

| | | Plant protein | Plant protein |
|---|---|---|---|
| | Control diet | replacement | replacement |
| | (J25 SCP | diet (50% | diet (100% |
| | meal-free) | replacement) | replacement) |
| Proximate | | | |
| Nutrient | | | |
| Dry matter (%) | 75.4 ± 0.4a | 78.0 ± 0.1b | 80.8 ± 0.2c |
| Protein (%) | 93.1 ± 0.2c | 92.2 ± 0.2b | 91.6 ± 0.2a |
| Energy (%) | 84.0 ± 0.4a | 85.0 ± 0.1b | 86.2 ± 0.2c |
| Essential amino | | | |
| acid (EAA) | | | |
| Arginine (%) | 96.0 ± 0.3a | 94.9 ± 0.3b | 94.5 ± 0.1b |
| Histidine (%) | 95.8 ± 0.3a | 93.3 ± 0.2c | 94.5 ± 0.7b |
| Isoleucine (%) | 92.9 ± 0.3a | 92.0 ± 0.4b | 90.2 ± 0.3c |
| Leucine (%) | 94.8 ± 0.2a | 93.7 ± 0.4b | 92.3 ± 0.2c |
| Lysine (%) | 96.3 ± 0.2a | 94.9 ± 0.4b | 92.1 ± 0.2c |
| Methionine (%) | 94.5 ± 0.3b | 98.3 ± 0.6a | 97.5 ± 0.2a |
| Phenylalanine (%) | 93.3 ± 0.3a | 91.1 ± 0.5b | 88.3 ± 0.5c |
| Threonine (%) | 93.9 ± 0.5a | 91.9 ± 0.8ab | 90.7 ± 1.3b |
| Tryptophan (%) | 99.5 ± 0.2ns | 99.5 ± 0.0 | 99.6 ± 0.1 |
| Valine (%) | 94.4 ± 0.3a | 93.6 ± 0.3b | 92.8 ± 0.1c |

¹Values within the same row having different letters are significantly different (P < 0.05). Values marked “ns” are not statistically different.

The 50 and 100% J25 SCP meal replacement test diets were digested significantly higher than the control diet for dry matter (78-81 vs. 75%; P<0.001), energy (85-86 vs. 84%; P<0.001) and methionine (98 vs. 95%; P<0.001) and at the same level for tryptophan (99-100%; P=0.679). Digestibility values for the test diets were slightly reduced for protein (92 vs. 93%; P<0.001) and other EAAs including arginine (95 vs. 96%; P<0.001), histidine (93-95 vs. 96%; P=0.001), isoleucine (90-92 vs. 93%; P<0.001), leucine (92-94 vs. 95%; P<0.001), lysine (92-95 vs. 96%; P<0.001), phenylalanine (88-91 vs. 93%; P<0.001), threonine (91-92 vs. 94%; P=0.015) and valine (93-94 vs. 94%; P<0.001).

The aforementioned studies evaluating M. capsulatus SCP meals had quite consistent crude protein contents of 68-73% (49-56% protein as expressed as ZAA); whereas the studies evaluating R. sphaeroides, A. marina, C. 320 ammoniagenes and M. extorquens were more variable (46-64% crude protein). The protein content of the J25 SCP 321 used in the present study exceeds all of these levels at 81% crude protein (65% ZAA). The ash composition of the J25 SCP meal used in this study (8%) is highly similar to those reported for M. capsulatus SCP meals (6-8%) and C. ammoniagenes SCP meal (10%); whereas those reported for M. extorquens SCP meals are lower (4%) and the levels reported for R. sphaeroides and A. marina are far higher (15-21%). Similarly, the crude lipid composition of the J25 SCP meal used in this study (10%) is similar to those reported for M. capsulatus SCP meals (8-11%) and C. ammoniagenes SCP meal (9%); whereas levels reported for M. extorquens, R. sphaeroides and A. marina SCP meals are far lower (<2%).

Meals produced from single-cell organisms such as yeast, bacteria, fungi and algae often contain higher levels (up to 20%) of non-protein nitrogen (NPN) than conventional feed ingredients. Some of the previously mentioned studies report NPN levels of 8-13%, which is highly congruent with the accepted levels for most bacteria of 8-12% (Demirbas and Demirbas 2011). The range of NPN levels observed for several test lots of J25 SCP meal are 3-6%, which indicates a lesser divergence between crude protein and true protein levels. Importantly, this suggests that J25 SCP meal would provide a negligible contribution to total dietary NPN levels at general feed inclusion levels. With a general proximate composition of 8% ash, 81% crude protein, 10% crude lipid, <1% carbohydrate and 21 MJ kg-1 gross energy, the J25 SCP meal used in this study is remarkably similar to conventional high-quality premium fish meals like anchovy meal and herring meal at 10-14% ash, 65-72% crude protein, 8-10% crude lipid, <1% carbohydrate and 20-21 MJ kg-1 gross energy (National Research Council (1993) Nutrient Requirements of Fish. National Academy Press, Washington, DC, USA, 114 p.; National Research Council (2011). Nutrient Requirements of Fish and Shrimp, National Academy Press, Washington, 376 pp.).

In contrast to premium fish meals, the J25 SCP meal and all of the plant-based protein feedstocks mentioned and poultry by-product meal, have lipid fractions that lack the physiologically essential LC-PUFA EPA+DHA. As mentioned, the lipid fraction of J25 SCP meal has a very simple profile, composed primarily (˜90%) of the saturated fatty acid C16:0 palmitic acid and the monounsaturated fatty acid C16:1n-7 palmitoleic acid. These particular fatty acids have been well digested (80-100%) by salmon pre-smolts based on a previous study using highly similar test diets (Tibbetts et al. Aquaculture 261, 1314-1327 2017) and should provide a relatively good source of digestible energy for the fish.

With a total amino acid content of 65% (composed of an almost 1:1 ratio of EAAs and NEAAs) and a high EAA index (0.9), J25 SCP meal is comparable or exceeds virtually all high-quality fish meals, poultry by-product meals and plant-protein concentrates commonly used in global salmon aquafeeds production. In addition, soy and corn-based protein products are deficient in lysine (corn protein) and methionine (soy protein), whereas J25 SCP meal contained these key EAAs.

The protein ADC value measured for J25 SCP meal with juvenile, pre-smolt stage Atlantic salmon in this study was 92%. This value is consistent (or higher) with the ADC values reported for typical high-quality salmon aquafeeds ingredients like premium fish meals (e.g., menhaden meal [83-88%], anchovy meal [91%], herring meal [91-95%]), poultry by-product meal (74-94%), corn gluten meal (92%), corn protein concentrate (91%), soybean meal (77-94%) and soy protein concentrate (90%) (Hajen et al. 1992; Sugiura et al. 1998; Glencross et al. 2004). Since the overall protein ADC for J25 SCP meal was high (92%), so too were the ADCs for its individual EAAs. The test diets contain a low level of fish meal (18%) with a focus on partial or complete SCP replacement of the major terrestrial plant-protein ingredients (from 31 to 0%). In this case, increasing levels of J25 SCP meal up to 26% of the diet generally led to no downward trend in dietary nutrient ADC

In summary, these examples have demonstrated the high potential for a novel low-trophic non-GMO single-cell protein (SCP) meal derived from methylotrophs, which can be produced at large scale in continuous-culture fermentation on inexpensive C1 methanol for sustainable farmed salmonid feed applications. The methylotrophic single cell protein product evaluated was high in crude protein and total amino acids; had a high EAA index and EAA:non-EAA ratio; and was dense in digestible calories. ADCs of proximate nutrients and EAAs were established for Atlantic salmon. Values were high for dry matter, protein, energy and EAAs; which led to the establishment of high values for protein digestibility-corrected amino acid scores (PDCAAS) and digestible indispensable amino acid scores (DIAAS). It was also demonstrated that nutritionally-balanced diets with partial or complete replacement of high-quality soy and corn proteins with the methylotrophic single cell protein product had no effect on diet palatability; as measured by feed consumption. In addition, dietary ADC values were increased for dry matter, energy and methionine at high inclusion levels of the methylotrophic single cell protein product.

### REFERENCES

Arru, B., Furesi, R., Gasco, L. Madau F. A., Pietro, P. (2019), The Introduction of Insect Meal Into Fish Diet: The First Economic Analysis on European Sea Bass Farming. Sustainability, 11(6), 1697.
Ebbinghaus, L. Ericsson, M., and Lindblom, M. (1981). The Production of Single Cell Protein from Methanol by Bacteria. Moo-Young, M. (Ed). Advances in Biotechnology Volume II Fuels, Chemicals, Foods and Waste Treatment. Elsevier Science Publishers.
MacLennan, D., Ousby, J., Owen, T., & Steer, D. (1974). Great Britian Patent GB 1,370,892. Mateles, R. I., & Battat, E. (1974). Continuous culture used for media optimization. Appl. Environ. Microbiol., 28(6), 901-905.
Goldberg, I. (1986). Single cell protein (Vol. 1). Springer Science & Business Media. Gow, J. S., Littlehailes, J. D., Smith, S. R. L., & Walter, R. B. (1975). SCP production from methanol: bacteria. In Single Cell Protein II, International Conference on Single Cell Protein. M.I.T. Press.
Rosenberg, Eugene, Edward F. DeLong, Stephen Lory, Erko Stackebrandt, and Fabiano Thompson, eds. (2014). The Prokaryotes: The Family Methylophilaceae. Berlin, Heidelberg: Springer Berlin Heidelberg.
Stringer, D. A. (1982). Industrial development and evaluation of new protein sources: micro-organisms. Proceedings of the Nutrition Society, 41(3), 289-300.
Southgate, G., & Goodwin, P. M. (1989). The regulation of exopolysaccharide production and of enzymes involved in C1 assimilation in Methylophilus methylotrophus. Microbiology, 135(11), 2859-2867.

### Example 7. Production Performance, Fish Health and Product Quality of Farmed Atlantic Salmon (Salmo salar L.) Fed Test Diets Containing Methylotrophic J25 Single-Cell Protein (SCP) Meal During the Juvenile Freshwater Grower Phase

As described herein, five (5) Atlantic salmon experimental test diets were formulated to contain varying levels of dietary J25 SCP meal to substitute for other conventional protein-rich aquafeed ingredients (e.g., fish, soy and corn protein). These test diets were then used in an 84-day feeding study with juvenile farmed Atlantic salmon to evaluate their effects on feed consumption, growth performance, nutrient utilization and fish health under controlled laboratory conditions.

An industry-representative control diet was formulated to satisfy the known dietary nutritional requirements of juvenile pre-smolt Atlantic salmon (Diet 1) according to NRC, Nutrient Requirements of Fish and Shrimp (2011). To evaluate the optimum dietary inclusion level of spray-dried J25 SCP meal in salmonid feeds, three (3) nutritionally-balanced diets were formulated with spray-dried J25 SCP meal incorporated at levels of 10% (Diet 2), 20% (Diet 3) and 30% (Diet 5). In parallel, in an effort to observe for any detectable effects on protein quality by spray-drying, a matching diet to that of Diet 3 (20% ‘spray-dried’ J25 SCP meal) was formulated to contain 20% ‘freeze-dried’ J25 SCP meal (Diet 4). The varying levels of dietary J25 SCP meals were incorporated into these test diet formulations at displacement (on a protein-to-protein basis) of conventional fish meal, soybean meal, soy protein concentrate and corn protein concentrate. All test diets were nutritionally-balanced to be isonitrogenous (50% crude protein), isolipidic (20% crude lipid) and isocaloric (19 MJ/kg digestible energy).

The experimental test diets were fed to 525 conventional (e.g., non-transgenic, diploid) juvenile pre-smolt Atlantic salmon (initial mean weight, 27.5±0.7 g) for 84 days at a water temperature of ±14.2±0.3° C. Freshwater circular in-flow rates to each research tank (100 L water volume) were held at 2-3 L/min in order to maintain dissolved oxygen (DO) saturation levels at >90%. Per diet, there were three tanks tested with each tank having 35 fish. To monitor the fish receiving each dietary treatment, all of the salmon in each research tank were batch-weighed at days 0, 28, 56 and 84. At each time point, randomly selected fish were analyzed for feed utilization.

TABLE 13
Proximate composition and amino acid profile
of the two J25 SCP meals (as-is basis)¹

| | J25 SCP MEALS | |
|---|---|---|
| | Freeze-Dried | Spray-Dried |
| Proximate Composition | | |
| Moisture % | 3.6 | 6.9 |
| Ash (%) | 11.5 | 11.6 |
| Crude protein, N × 6.25 (%) | 75.5 | 70.6 |
| True protein, Σ AA (%) | 47.2 | 49.9 |
| NPN (%)² | 4.5 | 3.3 |
| Crude lipid (%)³ | 0.3 | 1.3 |
| Carbohydrate (%)⁴ | 12.7 | 16.5 |
| Gross energy (MJ/kg) | 18.6 | 17.7 |
| Essential Amino Acid* | | |
| Arginine (%) (1.8) | 3.2 | 3.2 |
| Histidine (%) (0.8) | 1.0 | 1.0 |
| Isoleucine (%) (1.1) | 2.2 | 2.4 |
| Leucine (%) (1.5) | 3.9 | 4.1 |
| Lysine (%) (2.4) | 2.9 | 2.8 |
| Methionine (%) (0.7) | 1.2 | 1.3 |
| Methionine + Cysteine (%) | 1.4 | 2.2 |
| (1.1) | | |
| Phenylalanine (%) (0.9) | 2.2 | 2.4 |
| Phenylalanine + Tyrosine (%) | 4.3 | 4.6 |
| (1.8) | | |
| Threonine (%) (1.1) | 2.8 | 3.4 |
| Tryptophan (%) (0.3) | 1.0 | 0.9 |
| Valine (%) (1.2) | 3.0 | 3.3 |
| Non-essential amino acid | | |
| Alanine (%) | 3.8 | 4.3 |
| α-amino-N-butyric acid (%) | 0.1 | 0.1 |
| Aspartic acid (%) | 4.4 | 4.5 |
| Cysteine (%) | 0.2 | 1.0 |
| Glutamic acid (%) | 5.9 | 5.5 |
| Glycine (%) | 2.9 | 3.1 |
| Ornithine (%) | 0.2 | 0.3 |
| Proline (%) | 2.0 | 2.1 |
| Serine (%) | 2.1 | 2.0 |
| Taurine (%) | <0.1 | <0.1 |
| Tyrosine (%) | 2.1 | 2.3 |
| Σ EAA (%) | 23.3 | 24.8 |
| Σ NEAA (%) | 23.8 | 25.1 |
| Σ AA (%) | 47.2 | 49.9 |
| EAA:NEAA ratio | 1.0 | 1.0 |

*Essential amino acid requirements in parentheses after (%). Requirements according to NRC (2011)
¹Reported as % of sample unless otherwise stated
²Non-protein nitrogen estimated as total nitrogen − (true protein ÷ 6.25)
³Soxhlet extraction with petroleum ether
⁴Carbohydrate estimated as 100 − (crude protein + crude lipid + ash)

TABLE 14
Formulation of the five nutritionally-balanced experimental
test diets used to evaluate the effects of dietary inclusion
of spray-dried (SD) and freeze-dried (FD) J25 SCP meals on
growth performance, nutrient utilization and fish health
of juvenile Atlantic salmon (Salmo salar)

| | % Diet | Diet 2 | Diet 3 | Diet 4 | Diet 5 |
|---|---|---|---|---|---|
| | Diet 1 | (10% | (20% | (20% | (30% |
| Ingredient | (Control) | SD) | SD) | FD) | SD) |
| Fish meal, | 18.000 | 15.000 | 12.000 | 12.000 | 9.000 |
| herring/ | | | | | |
| anchovy | | | | | |
| (69% CP¹) | | | | | |
| Soybean meal | 15.000 | 10.000 | 5.000 | 5.000 | — |
| (48% CP) | | | | | |
| Corn protein | 12.000 | 8.000 | 4.000 | 4.000 | — |
| concentrate | | | | | |
| (78% CP) | | | | | |
| Soy protein | 9.000 | 6.000 | 3.000 | 3.000 | — |
| concentrate | | | | | |
| (67% CP) | | | | | |
| Spray-dried J25 | — | 10.000 | 20.000 | — | 30.000 |
| SCP meal (71% | | | | | |
| CP) | | | | | |
| Freeze-dried | — | — | — | 20.000 | — |
| J25 SCP meal | | | | | |
| (76% CP) | | | | | |
| Wheat gluten | 5.000 | 6.750 | 9.230 | 7.800 | 11.710 |
| meal (82% CP) | | | | | |
| Blood meal | 5.000 | 5.000 | 5.000 | 5.000 | 5.000 |
| (96% CP) | | | | | |
| Poultry by- | 4.000 | 4.000 | 4.000 | 4.000 | 4.000 |
| product meal | | | | | |
| (71% CP) | | | | | |
| Wheat flour | 7.210 | 11.090 | 13.960 | 15.360 | 16.830 |
| Fish Oil | 8.870 | 8.680 | 8.840 | 8.830 | 8.990 |
| Poultry fat | 4.435 | 4.430 | 4.420 | 4.415 | 4.495 |
| Canola oil | 4.435 | 4.430 | 4.420 | 4.415 | 4.495 |
| Calcium | 2.900 | 2.530 | 1.710 | 1.730 | 0.890 |
| phosphate, | | | | | |
| monobasic² | | | | | |
| L-Lysine | 1.940 | 1.960 | 1.970 | 2.000 | 2.000 |
| Vitamin and | 0.600 | 0.600 | 0.600 | 0.600 | 0.600 |
| Mineral | | | | | |
| mixture³ | | | | | |
| DL-Methionine | 0.400 | 0.450 | 0.520 | 0.520 | 0.600 |
| Choline | 0.400 | 0.400 | 0.400 | 0.400 | 0.400 |
| chloride | | | | | |
| Salt, NaCl | 0.250 | 0.250 | 0.250 | 0.250 | 0.250 |
| L-Tryptophan | — | 0.050 | 0.120 | 0.120 | 0.180 |
| Taurine | 0.500 | 0.500 | 0.500 | 0.500 | 0.500 |
| Vitamin C, | 0.030 | 0.030 | 0.030 | 0.030 | 0.030 |
| ascorbic acid | | | | | |
| ‘Stay-C 35’ | | | | | |
| Vitamin E, α- | 0.030 | 0.030 | 0.030 | 0.030 | 0.030 |
| tocopherol | | | | | |
| Total | 100.000 | 100.000 | 100.000 | 100.000 | 100.000 |

¹CP = Crude protein (N × 6.25)
²Ca(H₂PO₄)₂
³Corey Nutrition freshwater salmonid mixture

TABLE 15
Proximate composition of the five nutritionally-balanced experimental
test diets used to evaluate the effects of dietary inclusion
of spray-dried (SD) and freeze-dried (FD) J25 SCP meals on growth
performance, nutrient utilization and fish health of juvenile
Atlantic salmon (Salmo salar)

| | % Diet | Diet 2 | Diet 3 | Diet 4 | Diet 5 |
|---|---|---|---|---|---|
| Proximate | Diet 1 | (10% | (20% | (20% | (30% |
| Composition | (Control) | SD) | SD) | FD) | SD) |
| Moisture (%) | 5.9 | 5.8 | 5.9 | 5.2 | 5.8 |
| Ash (%) | 8.4 | 8.2 | 7.8 | 7.9 | 7.6 |
| Crude protein, | 49.8 | 48.9 | 49.1 | 49.0 | 49.5 |
| N × 6.25 (%) | | | | | |
| Crude lipid | 20.2 | 20.2 | 20.2 | 20.0 | 20.1 |
| (%)¹ | | | | | |
| Carbohydrate | 21.6 | 22.7 | 22.9 | 23.1 | 22.8 |
| (%)² | | | | | |
| Gross energy | 22.5 | 22.2 | 22.2 | 22.3 | 22.2 |
| (MJ/kg) | | | | | |

¹Soxhlet extraction with petroleum ether
²Carbohydrate estimated as 100 − (crude protein + crude lipid + ash)

TABLE 16
Overall growth performance and feed utilization of juvenile Atlantic salmon (Salmo salar)
fed five nutritionally-balanced experimental test diets containing varying dietary
inclusion of spray-dried (SD) and freeze-dried (FD) J25 SCP meals¹

| | Diet 1 | Diet 2 | Diet 3 | Diet 4 | Diet 5 | |
|---|---|---|---|---|---|---|
| | (Control) | (10% SD) | (20% SD) | (20% FD) | (30% SD) | P-value |
| Growth | | | | | | |
| Performance | | | | | | |
| Initial body | 27.7 ± 0.7ns | 26.9 ± 0.5 | 27.5 ± 0.8 | 28.0 ± 0.7 | 27.6 ± 0.9 | 0.484 |
| weight | | | | | | |
| (g/fish) | | | | | | |
| Final body | 117.2 ± 6.8a | 114.3 ± 7.6ab | 105.5 ± 7.4ab | 111.6 ± 5.7ab | 96.3 ± 8.1b | 0.034 |
| weight | | | | | | |
| (g/fish) | | | | | | |
| Weight gain | 89.5 ± 6.1a | 87.5 ± 7.3a | 78.0 ± 7.1ab | 83.7 ± 6.2ab | 68.7 ± 7.1b | 0.024 |
| (g/fish) | | | | | | |
| Fork length | 21.6 ± 0.5ns | 20.9 ± 1.4 | 19.8 ± 0.0 | 20.4 ± 0.2 | 20.2 ± 0.6 | 0.104 |
| (cm/fish)² | | | | | | |
| Fulton's | 1.3 ± 0.1ns | 1.3 ± 0.0 | 1.3 ± 0.1 | 1.3 ± 0.0 | 1.2 ± 0.0 | 0.063 |
| condition | | | | | | |
| factor | | | | | | |
| (g/cm³)² | | | | | | |
| Specific | 1.4 ± 0.1a | 1.4 ± 0.1a | 1.2 ± 0.1ab | 1.3 ± 0.1ab | 1.1 ± 0.1b | 0.008 |
| growth rate | | | | | | |
| (%/day) | | | | | | |
| Survival (%) | 100 | 100 | 100 | 100 | 100 | — |
| Feed | | | | | | |
| Utilization | | | | | | |
| Apparent | 81.2 ± 1.8a | 83.5 ± 2.7a | 77.8 ± 1.9a | 80.1 ± 3.0a | 69.9 ± 2.0b | <0.001 |
| feed intake | | | | | | |
| (g/fish) | | | | | | |
| Feed | 0.9 ± 0.0ns | 1.0 ± 0.1 | 1.0 ± 0.1 | 1.0 ± 0.0 | 1.0 ± 0.1 | 0.328 |
| conversion | | | | | | |
| ratio (g | | | | | | |
| feed/g gain) | | | | | | |
| Protein intake | 40.4 ± 0.9a | 40.8 ± 1.3a | 38.2 ± 1.0a | 39.3 ± 1.5a | 34.6 ± 1.0b | <0.001 |
| (g/fish) | | | | | | |
| Protein | 2.1 ± 0.1ns | 2.0 ± 0.1 | 1.9 ± 0.2 | 2.0 ± 0.1 | 1.9 ± 0.2 | 0.368 |
| efficiency | | | | | | |
| ratio (g | | | | | | |
| gain/g DM | | | | | | |
| protein | | | | | | |
| intake) | | | | | | |

¹Values within the same row having different letters are significantly different (P < 0.05). Values marked “ns” are not statistically different.
²Values for initial fish (Day 0) were as follows: Fork length, (12.9 ± 1.1 cm/fish); Fulton's condition factor, 1.2 ± 0.1 g/cm3; Haematocrit value, 69.1 ± 4.1%3; Hepatosomatic index, 1.3 ± 0.2%; Viscerosomatic index, 9.3 ± 0.9% (n = 35).

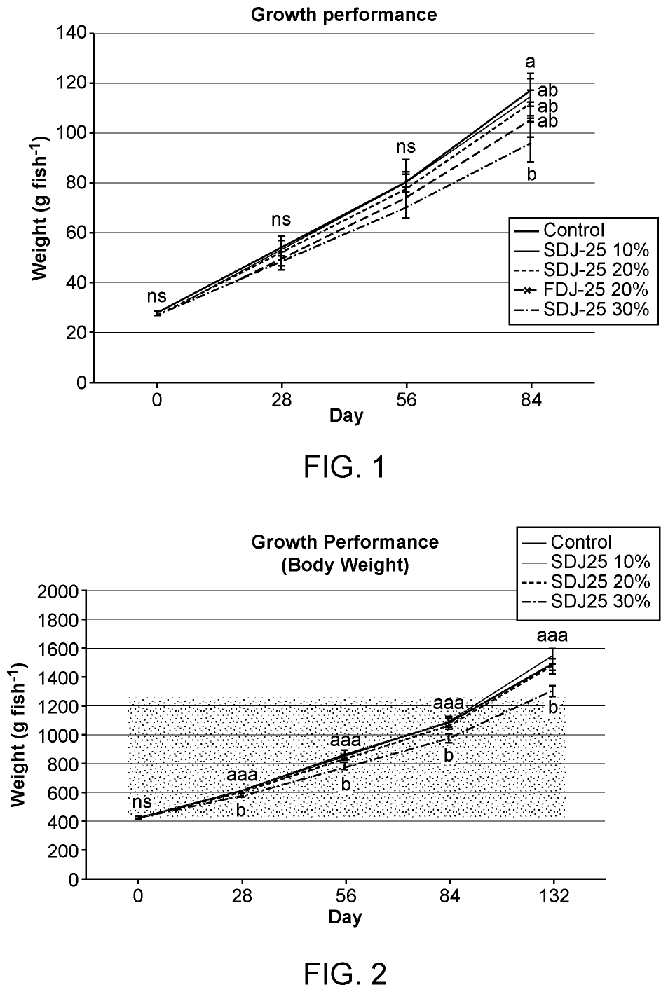

The results of the juvenile study indicate there is 100% survivability with positive results across all health metrics (Table 16). There is no statistical difference in performance of freeze dried product when compared to spray dried (Tables 13-15). The growth performance of the fish was equal to control at up to 20% inclusion as shown in FIG. 1. At 30% inclusion, the digestibility and conversion efficiency was just as strong as control. In addition, the health results for all diets was comparable to the control diet as determined by intestinal histomorphology and pathology of juvenile salmon (Table 16). Therefore, methylotrophic single cell protein products can be used to replace animal and plant-based protein and be fed to juvenile fish without negatively impacting juvenile growth or health.

### Example 8. Production Performance, Fish Health and Product Quality of Farmed Atlantic Salmon (Salmo salar L.) Fed Test Diets Containing Methylotrophic J25 Single-Cell Protein (SCP) meal during the seawater grower phase

This example describes analysis of feeding SCP diets to larger fish during the “post-smolt” seawater phase of the production cycle, including the measurement of product quality parameters important for fish closer to industrial harvest size. As such, a large scaled-up sample (121 kg) of spray-dried J25 SCP meal was generated for this project and was first characterized for its proximate composition, amino acid profile and elemental concentrations. As in example 7, four Atlantic salmon experimental test diets were formulated to contain a constant low fish meal inclusion level with varying levels of dietary spray-dried J25 SCP meal to partially or completely substitute predominately for soy protein. These test diets were then used in a 132-day feeding study with post-smolt farmed Atlantic salmon in the seawater production phase to evaluate their effects on feed consumption, growth performance, nutrient utilization, fish health and final product quality.

An industry-representative control diet (containing 0% J25 SCP meal) was formulated to satisfy the dietary nutritional requirements of Atlantic salmon (Diet A) according to NRC (2011). To evaluate the optimum dietary inclusion level of J25 SCP meal in seawater-phase Atlantic salmon feeds in low fish meal feeds (constant 15% in all diets), three isonitrogenous, isolipidic, isocarbohydric and isocaloric diets were formulated with increasing J25 SCP meal incorporation levels of 10% (Diet B), 20% (Diet C) and 30% (Diet D). The varying dietary J25 SCP meal levels were achieved predominantly by partial (33 or 66%) or complete (100%) displacement (on a protein-to-protein basis) of conventional soy protein concentrate (24, 16, 8 and 0%). To balance the test diets, it was possible to achieve modest reductions in the use of wheat gluten meal (2.7 to 0.5%), blood meal (10.0 to 0.1%), calcium monophosphate (3.7 to 0%) and DL-methionine (0.2 to 0%); increased use of wheat flour (11-21%); with only minimal (<1%) requirement for supplemental L-Lysine and L-Histidine.

The experimental test diets were fed to 420 conventional (e.g., non-transgenic, diploid) Saint John River strain post-smolt Atlantic salmon (initial mean weight, 423.7±3.8 g) for 132 days in a marine recirculating aquaculture system (RAS) at a stable water temperature of +13.8±0.4° C. Seawater (25±3 ppt salinity) with counter-clockwise circular in-flow rates to each research tank (1,200 L water volume) were held at 20 L/min in order to maintain dissolved oxygen (DO) saturation levels at >90%.

On Day 0, fish in all research tanks were individually weighed in order to document the initial mean weight of the fish in each tank. Per diet, there were three tanks tested each tank having 35 fish. To monitor the fish receiving each dietary treatment, all of the salmon in each research tanks were batched-weighed at days 0, 28, 56, 84 and 132. At each time point, randomly selected fish were analyzed for feed utilization.

TABLE 17
Formulation of the four nutritionally-balanced experimental
test diets used to evaluate the effects of dietary inclusion
of spray-dried J25 SCP meal on production performance, fish health
and product quality of Atlantic salmon (Salmo salar L.)
during the seawater grower phase (as-fed basis)

| | % of Diet | | | |
|---|---|---|---|---|
| | Diet A | Diet B | Diet C | Diet D |
| | (Control) | (10% SD) | (20% SD) | (30% SD) |
| Ingredient | | | | |
| Fish meal (71% | 15.000 | 15.000 | 15.000 | 15.000 |
| CP) | | | | |
| Poultry by- | 10.000 | 10.000 | 10.000 | 10.000 |
| product meal | | | | |
| (67% CP) | | | | |
| Spray-dried J25 | — | 10.000 | 20.000 | 30.000 |
| SCP meal (75% | | | | |
| CP) | | | | |
| Soy protein | 24.000 | 16.000 | 8.000 | — |
| concentrate | | | | |
| (61% CP) | | | | |
| Blood meal | 9.980 | 7.030 | 3.720 | 0.090 |
| (94% CP) | | | | |
| Wheat gluten | 2.710 | 2.010 | 1.450 | 0.490 |
| meal (79% CP) | | | | |
| Wheat flour | 10.970 | 14.045 | 17.305 | 21.045 |
| (15% CP) | | | | |
| Fish oil | 14.420 | 14.130 | 13.830 | 13.550 |
| Canola oil | 7.210 | 7.065 | 6.915 | 6.775 |
| Soy lecithin | 1.000 | 1.000 | 1.000 | 1.000 |
| Calcium | 3.680 | 2.450 | 1.220 | — |
| phosphate, | | | | |
| monobasic | | | | |
| Choline | 0.400 | 0.400 | 0.400 | 0.400 |
| chloride | | | | |
| Vitamin and | 0.300 | 0.300 | 0.300 | 0.300 |
| Mineral mixture | | | | |
| L-Lysine | — | 0.320 | 0.580 | 0.900 |
| DL-Methionine | 0.190 | 0.110 | 0.040 | — |
| L-Histidine | — | — | 0.100 | 0.310 |
| Vitamin C, | 0.030 | 0.030 | 0.030 | 0.030 |
| ascorbic acid | | | | |
| ‘Stay-C 35’ | | | | |
| Vitamin E, α- | 0.030 | 0.030 | 0.030 | 0.030 |
| tocopherol | | | | |
| Carophyll pink | 0.080 | 0.080 | 0.080 | 0.080 |
| Total | 100.000 | 100.000 | 100.000 | 100.000 |
| Calculated | | | | |
| Composition | | | | |
| Crude protein | 45 | 45 | 45 | 45 |
| (%) | | | | |
| Crude lipid (%) | 26 | 26 | 26 | 26 |
| Carbohydrate | 18 | 18 | 18 | 18 |
| (%) | | | | |
| Digestible | 20 | 20 | 19 | 19 |
| energy (MJ/kg) | | | | |
| Phosphorous | 1.7 | 1.7 | 1.7 | 1.7 |
| (%) | | | | |
| EPA + DHA (%) | 2.0 | 2.0 | 2.0 | 1.9 |
| Astaxanthin | 80 | 80 | 80 | 80 |
| (mg/kg) | | | | |
| Lysine (%) | 3.3 | 3.3 | 3.3 | 3.3 |
| Methionine (%) | 1.0 | 1.0 | 1.0 | 1.0 |

TABLE 18
Overall growth performance and feed utilization of Atlantic salmon (Salmo salar
L.) fed four nutritionally-balanced experimental test diets containing varying dietary
inclusion of spray-dried J25 SCP meal during the seawater grower phase¹

| | Diet A | Diet B (10% | Diet C (20% | Diet D (30% | |
|---|---|---|---|---|---|
| | (Control) | J25) | J25) | J25) | P-value |
| Growth | | | | | |
| Performance | | | | | |
| Initial body | 424.3 ± 2.1ns | 423.5 ± 2.7 | 423.4 ± 7.7 | 423.5 ± 2.9 | 0.994 |
| weight | | | | | |
| (g/fish) | | | | | |
| Final body | 1485.4 ± 42.1a | 1543.6 ± 54.4a | 1475.3 ± 54.9a | 1302.3 ± 36.0b | 0.001 |
| weight | | | | | |
| (g/fish) | | | | | |
| Weight gain | 1061.2 ± 43.2a | 1120.1 ± 56.6a | 1052.0 ± 60.0a | 878.8 ± 34.0b | 0.002 |
| (g/fish) | | | | | |
| Weight gain | 350 ± 11a | 365 ± 15a | 349 ± 17a | 308 ± 7b | 0.004 |
| (% of IBW) | | | | | |
| Fork length | 46.5 ± 0.5ns | 47.2 ± 0.6 | 47.1 ± 0.3 | 46.6 ± 1.1 | 0.550 |
| (cm/fish)² | | | | | |
| Fulton's | 1.4 ± 0.0ns | 1.4 ± 0.0 | 1.4 ± 0.0 | 1.3 ± 0.0 | 0.166 |
| condition | | | | | |
| factor | | | | | |
| (g/cm³)² | | | | | |
| Specific | 0.95 ± 0.02a | 0.98 ± 0.03a | 0.95 ± 0.04a | 0.85 ± 0.02b | 0.003 |
| growth rate | | | | | |
| (%/day) | | | | | |
| Thermal | 0.21 ± 0.01a | 0.22 ± 0.01a | 0.21 ± 0.01a | 0.19 ± 0.0b | 0.002 |
| growth | | | | | |
| coefficient | | | | | |
| (g⅓/° C. · d) | | | | | |
| Survival (%) | 100 | 100 | 99 | 100 | — |
| Feed | | | | | |
| Utilization | | | | | |
| Apparent feed | 1051.3 ± 80.4a | 1092.4 ± 57.5a | 1016.0 ± 83.3ab | 864.7 ± 11.8b | 0.012 |
| intake (g/fish) | | | | | |
| Feed | 0.99 ± 0.04ns | 0.98 ± 0.01 | 0.97 ± 0.05 | 0.98 ± 0.03 | 0.842 |
| conversion | | | | | |
| ratio (g feed/g | | | | | |
| gain) | | | | | |
| Protein intake | 533.2 ± 40.8a | 560.9 ± 29.5a | 505.5 ± 41.4ab | 425.7 ± 5.8b | 0.005 |
| (g/fish) | | | | | |
| Protein | 2.04 ± 0.24ns | 1.75 ± 0.22 | 2.11 ± 0.44 | 2.34 ± 0.30 | 0.228 |
| efficiency | | | | | |
| ratio³ | | | | | |

¹Values within the same row having different superscript letters are significantly different (P < 0.05). Values marked “ns” are not statistically different.
²Values for initial fish (Day 0) were as follows: Fork length, 33.9 ± 0.9 cm/fish; Fulton's condition factor, 1.1 ± 0.1 g/cm3; Hepatosomatic index, 1.0 ± 0.1%; Viscerosomatic index, 7.9 ± 0.7% (n = 10)
³PER = (g wet weight gain/g DM protein intake)

TABLE 19
Protein deposition rate and utilization efficiency of Atlantic salmon (Salmo salar
L.) fed four nutritionally-balanced experimental test diets containing varying dietary
inclusion of spray-dried J25 SCP meal during the seawater grower phase¹

| | Diet A | Diet B (10% | Diet C (20% | Diet D (30% | |
|---|---|---|---|---|---|
| | (Control) | J25) | J25) | J25) | P-value |
| Protein intake | 533.2 ± 40.8a | 560.9 ± 29.5a | 505.5 ± 41.4ab | 425.7 ± 5.8b | 0.005 |
| (g/fish) | | | | | |
| Protein gain | 201.4 ± 10.9a | 214.6 ± 8.9a | 195.3 ± 12.4ab | 169.4 ± 9.9b | 0.005 |
| (g/fish)² | | | | | |
| Protein | 109.8 ± 6.0a | 117.0 ± 4.9a | 106.4 ± 6.7ab | 92.3 ± 5.4b | 0.005 |
| deposition | | | | | |
| rate | | | | | |
| (mg/° C. · d)³ | | | | | |
| Apparent net | 37.8 ± 1.4ns | 38.3 ± 1.6 | 38.7 ± 1.0 | 39.8 ± 1.9 | 0.474 |
| protein | | | | | |
| retention (%) | | | | | |

¹Values within the same row having different superscript letters are significantly different (P < 0.05). Values marked “ns” are not statistically different.
²Wet weight basis
³Calculated according to Dumas et al., Aquaculture 492: 24-34 (2018)

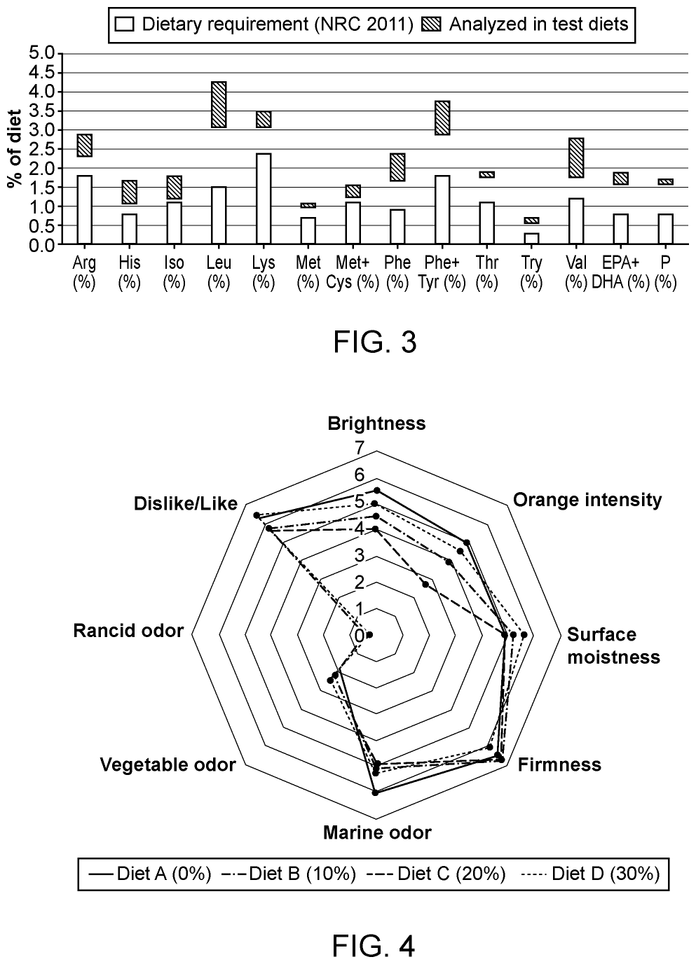

These results show equal growth performance at 20% inclusion and trending improved growth at 10% inclusion. Specifically, FIG. 2 shows 10% SCP inclusion trending improved growth over control, anticipated to become statistically significant over typical grow out phase (additional 6-12 months) whereas 20% SCP inclusion is statistically equivalent to commercial control and 30% SCP inclusion demonstrates decline in feed in-take, resulting in lower weight gain. However, 30% SCP inclusion did not negatively impact fish health. Table 18 shows a feed conversion ratio of <1.0 for all test diets and FIG. 3 shows test diets contain more essential amino acid, essential n-3 LC-PUFA (EPA+DHA) and phosphorous content than published dietary requirements NRC (2011). Table 19 shows increased protein gain and protein deposition rate observed with fish fed diet with 10% inclusion of SCP protein (J25). There was no difference in key fish health metrics across all test diets, including gut health, whole blood, plasma biochemistry, and analyte concentration. Finally, FIG. 4 shows in a blind taste panel preferred the taste of fillets from salmon fed diets containing 20% and 30% SCP and rated the fillets as “brighter” in appearance and less fishy smelling when compared to control diet. Therefore, methylotrophic single cell protein products can be used to replace animal and plant-based protein in animal feed to produce commercially viable fish.

In summary, Examples 7 and 8, demonstrate methylotrophic single cell protein products were successfully used to replace animal-based and plant-based protein in animal feed.

1. An animal feed comprising at least 5% methylotrophic single cell protein product and less than 20% plant-based protein products and less 35% animal-derived protein products, wherein the methylotrophic single cell protein product comprises a methanol fed methylotroph and wherein the methylotroph is not genetically modified.

2. The animal feed of claim 1, wherein the animal feed comprises at least 10%, 15%, 20%, 25%, or 30% methylotrophic single cell protein.

3. The animal feed of claim 1, wherein the animal feed comprises less than 30%, 20%, 15%, or 10% animal-derived protein products.

4. An animal feed comprising at least 5% methylotrophic single cell protein and less than 40% plant-based protein products and less than 40% animal-derived protein products, wherein the methylotrophic single cell protein product comprises a methanol fed methylotroph of the genus Methylovorus and wherein the methylotroph is not genetically modified.

5. An animal feed comprising a methylotrophic single cell protein product comprising all essential amino acids, wherein the methylotrophic single cell protein product comprises a methanol fed methylotroph that is not genetically modified, and wherein the apparent digestibility coefficient for each essential amino acid of the single cell protein product is at least 85% in Atlantic salmon.

6. The animal feed of claim 1, wherein the Essential Amino Acid Index in the single cell protein product is least 0.9 for Atlantic salmon.

7. The animal feed of claim 1, wherein the animal feed is an aquafeed.

8. The animal feed of claim 7, wherein the aquafeed is feed for carnivorous fish.

9. The animal feed of claim 8, wherein the carnivorous fish is a salmonid.

10. The animal feed of claim 1, wherein the methylotrophic single cell protein is the primary protein in the feed.

11. The animal feed of claim 1, wherein the animal feed comprises 20% to 30% methylotrophic single cell protein.

12. The animal feed of claim 1, wherein the animal feed comprises 10% to 30% methylotrophic single cell protein.

13. The animal feed of claim 1, wherein the animal feed comprises 1% to 20% fish meal.

14. The animal feed of claim 1, wherein the methylotroph is not fed methane.

15. The animal feed of claim 1, wherein the methylotroph is a methylovorus methylotroph.

16. A methylotrophic single cell protein product comprising all essential amino acids, wherein the methylotrophic single cell protein product comprises a methanol fed methylotroph, wherein the methylotroph is not genetically modified, and wherein the apparent digestibility coefficient for each essential amino acid in the methylotrophic single cell protein product is at least 85% in Atlantic salmon.

17. The methylotrophic single cell protein product of claim 16, wherein the Essential Amino Acid Index in the single cell protein product is least 0.9 for Atlantic salmon.

18. The methylotrophic single cell protein product of claim 16, wherein the methylotrophic single cell protein product comprises a crude protein content of greater than 50%, 60%, 70%, 80%, or 90%.

19. The methylotrophic single cell protein product of claim 16, wherein the methylotrophic single cell protein product comprising a carbohydrate content of 1% to 15%.

20. The methylotrophic single cell protein product of claim 16, wherein the methylotroph is of the Methylovorus genus.

21. A method of feeding fish comprising feeding to the fish the animal feed of claim 1.

22. The method of claim 22, wherein the fish are carnivorous fish.
